SEQUENCE LISTING (7 sequences)

SEQ ID NO: 1 - DNA, 1284 bp, Homo sapiens
  JNK (Accession NM_139049) (1)..(1284)
atgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatga

SEQ ID NO: 2 - protein, 427 aa, Homo sapiens
  JNK (Accession # NP_620637) (1)..(427)
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 3 - DNA, 1842 bp, Homo sapiens
  AIF (Accession # AF100928) (1)..(1842)
atgttccggtgtggaggcctggcggcgggtgctttgaagcagaagctggtgcccttggtgcggaccgtgtgcgtccgaagcccgaggcagaggaaccggctcccaggcaacttgttccagcgatggcatgttcctctagaactccagatgacaagacaaatggctagctctggtgcatcagggggcaaaatcgataattctgtgttagtccttattgtgggcttatcaacagtaggagctggtgcctatgcctacaagactatgaaagaggacgaaaaaagatacaatgaaagaatttcagggttagggctgacaccagaacagaaacagaaaaaggccgcgttatctgcttcagaaggagaggaagttcctcaagacaaggcgccaagtcatgttcctttcctgctaattggtggaggcacagctgcttttgctgcagccagatccatccgggctcgggatcctggggccagggtactgattgtatctgaagatcctgagctgccgtacatgcgacctcctctttcaaaagaactgtggttttcagatgacccaaatgtcacaaagacactgcgattcaaacagtggaatggaaaagagagaagcatatatttccagccaccttctttctatgtctctgctcaggacctgcctcatattgagaatggtggtgtggctgtcctcactgggaagaaggtagtacagctggatgtgagagacaacatggtgaaacttaatgatggctctcaaataacctatgaaaagtgcttgattgcaacaggaggtactccaagaagtctgtctgccattgatagggctggagcagaggtgaagagtagaacaacgcttttcagaaagattggagactttagaagcttggagaagatttcacgggaagtcaaatcaattacgattatcggtgggggcttccttggtagcgaactggcctgtgctcttggcagaaaggctcgagccttgggcacagaagtgattcaactcttccccgagaaaggaaatatgggaaagatcctccccgaatacctcagcaactggaccatggaaaaagtcagacgagagggggttaaggtgatgcccaatgctattgtgcaatccgttggagtcagcagtggcaagttacttatcaagctgaaagacggcaggaaggtagaaactgaccacatagtggcagctgtgggcctggagcccaatgttgagttggccaagactggtggcctggaaatagactcagattttggtggcttccgggtaaatgcagagctacaagcacgctctaacatctgggtggcaggagatgctgcatgcttctacgatataaagttgggaaggaggcgggtagagcaccatgatcacgctgttgtgagtggaagattggctggagaaaatatgactggagctgctaagccgtactggcatcagtcaatgttctggagtgatttgggccccgatgttggctatgaagctattggtcttgtggacagtagtttgcccacagttggtgtttttgcaaaagcaactgcacaagacaaccccaaatctgccacagagcagtcaggaactggtatccgatcagagagtgagacagagtccgaggcctcagaaattactattcctcccagcaccccggcagttccacaggctcccgtccagggggaggactacggcaaaggtgtcatcttctacctcagggacaaagtggtcgtggggattgtgctatggaacatctttaaccgaatgccaatagcaaggaagatcattaaggacggtgagcagcatgaagatctcaatgaagtagccaaactattcaacattcatgaagactga

SEQ ID NO: 4 - protein, 613 aa, Homo sapiens
  AIF (Accession # AAD16436) (1)..(613)
MFRCGGLAAGALKQKLVPLVRTVCVRSPRQRNRLPGNLFQRWHVPLELQMTRQMASSGASGGKIDNSVLVLIVGLSTVGAGAYAYKTMKEDEKRYNERISGLGLTPEQKQKKAALSASEGEEVPQDKAPSHVPFLLIGGGTAAFAAARSIRARDPGARVLIVSEDPELPYMRPPLSKELWFSDDPNVTKTLRFKQWNGKERSIYFQPPSFYVSAQDLPHIENGGVAVLTGKKVVQLDVRDNMVKLNDGSQITYEKCLIATGGTPRSLSAIDRAGAEVKSRTTLFRKIGDFRSLEKISREVKSITIIGGGFLGSELACALGRKARALGTEVIQLFPEKGNMGKILPEYLSNWTMEKVRREGVKVMPNAIVQSVGVSSGKLLIKLKDGRKVETDHIVAAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPPSTPAVPQAPVQGEDYGKGVIFYLRDKVVVGIVLWNIFNRMPIARKIIKDGEQHEDLNEVAKLFNIHED

SEQ ID NO: 5 - DNA, 21 bp, Homo sapiens
  JNK RNAi SEQ1 (1)..(21)
aagcaccttcattctgctgga

SEQ ID NO: 6 - DNA, 21 bp, Homo sapiens
  JNK RNAi SEQ2 (1)..(21)
aagcggatctctgtagacgaa

SEQ ID NO: 7 - DNA, 19 bp, Homo sapiens
  AIF RNAi SEQ1 (1)..(19)
gtagccaaactattcaaca

(19) United States
(12) Patent Application Publication
(10) Pub. No.: US 2007/0003531 A1
(43) Pub. Date: Jan. 4, 2007

(54) Methods for improving immunotherapy by enhancing survival of antigen-specific cytotoxic T lymphocytes

(75) Inventors: Bijay Mukherji, Farmington, CT (US); Shikhar Mehrotra, West Hartford, CT (US); Arvind Chhabra, West Hartford, CT (US)

Correspondence Address: MCCARTER & ENGLISH LLP;CITYPLACE I, 185 ASYLUM STREET, HARTFORD, CT, 06103, US

(73) Assignee: University of Connecticut

(21) Appl. No.: 11/479,078

(22) Filed: Jun. 30, 2006

Related U.S. Application Data
(60) Provisional application No. 60/695,720, filed on Jun. 30, 2005.

(51) Int. Cl. A61K 48/00; A61K 31/4162

(52) U.S. Cl. 424/93.21; 514/44; 514/406

(57) ABSTRACT

The invention relates generally to methods for enhancing immunity by improving the survival of activated T cells comprising the administration to a cell or a subject in need thereof an effective amount of an inhbitor of JNK and/or AIF. In other aspects the invention relates to the administration of an effective amount of an enzymatic nucleic acid, a pyrazoloanthrone or derivative, or combinations thereof to reduce activation induced cell death (AICD), programmed cell death (PCD), or both of antigen specific T cells.

### CROSS-REFERENCE TO RELATED APPLICATIONS

Under 35 U.S.C. §119(e) this application claims priority to, and the benefit of U.S. Provisional Application No. 60/695,720 filed Jun. 30, 2005.

### INCORPORATION BY REFERENCE

In compliance with 37 C.F.R. §1.52(e)(5), the sequence information contained on compact disc, file name: 98121.00137SEQLIST.ST25; created on: Jun. 28, 2006; size: 14 KB; is hereby incorporated by reference in its entirety. The Sequence Listing information recorded in computer readable form is identical to the written Sequence Listing provided herewith.

### FIELD OF THE INVENTION

The invention relates generally to methods for treating cancer comprising administering an effective amount of an inhibitor of JNK expression or activity, to reduce activation induced cell death (AICD), programmed cell death (PCD), or both of an antigen or epitope specific cytotoxic T lymphocyte (CTL) cell. The invention also relates to CTLs generated according to the methods of the invention for use in treating cancer, for example, in active immunization or adoptive transfer treatments.

### BACKGROUND

Active immunization with tumor associated peptides as well as adoptive transfer of ex vivo expanded tumor epitope specific cytolytic T lymphocytes (CTL) are two widely pursued approaches toward specific immunotherapy for cancer. The overall results of both approaches have been modest but encouraging, and intense efforts are underway to make them more effective. In this context, the generation of a robust and long-lived anti-tumor T cell response remains an important objective. Although tumor epitope-specific CTLs can be activated and expanded with appropriate stimuli—in vitro as well as in vivo—programmed cell death (PCD) of activated T cells can be a major impediment in orchestrating a robust and long-lived anti-tumor CTL response through immunization or adoptive transfer of CTLs.

For example, much interest has been generated in cancer vaccine therapy with specific peptides, protein antigens, DNA, etc. Most of these immunogens are “self” antigens, yet many cancer patients as well as normal healthy hosts harbor precursor CTLs for such “self” epitopes. Ex vivo stimulation of T cells as well as in vivo immunization with such “self” peptides or tumor associated antigens (TAAs) lead to the activation and expansion of the antigen specific CTLs. These TAA specific T cells are susceptible to PCD. As indicated above, PCD in activated T cells in tumor immunotherapy can be counterproductive, particularly if the activated T cells undergo apoptotic death after the second encounter with the specific epitope.

In 1987 Ashwell et al. first noted growth arrest in T cell hybridoma cells upon T cell receptor (TCR)-mediated stimulation. Shi et al. (1989) thereafter showed that TCR-driven signaling leads to death of the cells and coined the term activation induced cell death (AICD). It is now known that T cell responses follow three phases: amplification, contraction, and memory generation. However, neither the extent of AICD in these self-but-tumor-associated antigen (TAA) reactive primary CTLs nor the feasibility of rescuing them from AICD has been carefully examined.

During the contraction phase of a primary T cell response, the majority of the antigen specific T cells undergo apoptotic death as a form of PCD primarily to maintain homeostasis. AICD, in contrast, involves the apoptotic deletion of a significant fraction of the activated population after an effector response, i.e., upon secondary encounter of antigens. In AICD, the effector function and the death are, paradoxically, triggered by T cell receptor (TCR)-driven signaling (i.e., activation induced). In any event, both processes (i.e., PCD and AICD) are designed to serve useful purposes by limiting the expansion of activated CD8⁺ or cytotoxic T lymphocytes and precluding the detrimental side effects of a continued and uncontrolled effector response.

There is widespread belief that the principal trigger for AICD is initiated through the death receptors (DRs), FasR and the TNF family of receptors, and that the cell death is mediated by the eventual activation of caspases. A universal role of the Fas and TNF family receptor-driven signaling for T cell apoptosis has, however, been controversial and both caspase-dependent and caspase-independent deaths in T cells have been described. For example, it has been shown that the death signal for T cells and especially for CD8+ CTLs can be triggered internally and death can occur without the involvement of caspases. Thus, it is now becoming increasingly apparent that an intrinsic cell death pathway triggers AICD in cytotoxic T lymphocytes without involving the extrinsic death receptors such as Fas and/or TNF family receptors.

The death receptor (DR)-driven extrinsic apoptotic pathways and the DR-independent instrinsic pathways may converge on mitochondria, and activate both caspase-dependent and caspase-independent mitochondria-based death machineries. The release of mitochondrial apoptogenic proteins such as cytochrome c can induce caspase-dependent cell death by activation of caspase-3. Mitochondria also sequester apoptosis inducing factor (AIF) and endonuclease G, both of which cause DNA fragmentation and cell death in a caspase-independent manner. Upon release from the mitochondria, AIF translocates to the nucleus and causes large-scale DNA fragmentation.

The c-jun terminal kinase, JNK, is a member of the MAP kinase family of signaling proteins. JNK has been associated with a variety of biological processes most notably in inflammation and transformation. All three MAP kinases (i.e., p38, JNK and ERK) have been found to have different roles in T cell biology such as in proliferation, effector function, survival, as well as death. Different isoforms of JNK have been found to have divergent roles in CD4⁺ and CD8⁺ T cells. For example, JNK1 is needed for the expression of IL-2Rα (CD25) upon activation, and for proliferation while JNK2 down regulates IL-2 production in CD 8+T cells. As such, jnk-1-null (jnk1 −/−) mice exhibit marked reduction in the expansion of antigen-driven CD8⁺ T cells.

JNK has also been implicated in the negative selection of thymoctes. As such, both JNK1 and JNK2 have been found to have stage-dependent roles in T cell development. For example, while JNK1 has been associated with anti-CD3 induced apoptosis of double positive thymocytes, jnk2 null double positive thymocytes are resistant to anti-CD3 induced apoptosis although they are sensitive to apoptosis induced by Dexamethasone, anti-Fas antibody or UV radiation. Thus, immature thymocytes seem to need one form of JNK or another for undergoing receptor-driven apoptosis but mature T cells need JNK1 for proliferation and IL-2 synthesis. Furthermore, the immature thymocytes do not seem to need JNK2 to undergo AICD. These observations suggest a role for JNK in apoptosis in immature thymocytes but not in mature T cells, however, our studies reveal that JNK is an important player in apoptotic death in CTLs in the periphery.

JNK is activated by dual phosphorylation of the Thr-183 and Tyr-185 residues within its Thr-Pro-Tyr motif and it can be activated by cytokines and environmental stress. Its activation leads to the up regulation of c-jun and eventual phosphorylation of transcription factors such as AP-1, and other proteins, some of which are associated with apoptosis. Death in CD 8⁺ T cells is believed to be driven by the internal apoptotic pathway, and results from mitochondrial dysfunction, the release of reactive oxygen intermediates, and stress. Also, as JNK can play a role in stress-induced activation of the cytochrome c-mediated cell death pathway, we posited that it is likely that JNK is involved in AICD in antigen specific CD 8⁺ CTLs.

Our studies show that that Melan-A/MART-1₂₇₋₃₅ epitope-specific CTLs expanded in an in vitro dendritic cell (DC)-based stimulation protocol undergo apoptotic death on repetitive stimulation by immature as well as by fully activated DCs. Similarly, a large fraction of MART-1₂₇₋₃₅ epitope-specific CTLs undergo AICD upon the secondary encounter of the cognate epitope. In addition, we show that AICD in CTL cells involves large-scale (˜50 kb) DNA fragmentation associated with the mitochondrion-nuclear translocation of AIF. Moreover, our results show that during AICD in CTLs, caspase-8 and caspase-3 are not activated, cytochrome-c is not released, and cell death is not associated with oligosomal DNA fragmentation. However, treatment with a JNK inhibitor blocks the mitochondrion-nuclear translocation of AIF and prevents AICD in these CTLs. The ability to rescue these CTLs would provide a new and useful means for treating cancer.

Therefore, compositions and methods that interfere with the “premature” AICD of tumor antigen specific T cells will improve the duration and effectiveness of active specific vaccination and adoptive immunotherapy. Specifically, such compositions and methods will be effective for the treatment of cancer by reducing antigen activated CTL cell death, and increasing CTL populations.

### SUMMARY OF THE INVENTION

The present invention relates to the discovery that disruption of the cellular processes that culminate in the mitochondrion-nuclear translocalization of AIF can enhance the survival rate of CTLs. In particular, we demonstrate that epitope specific primary CTLs undergo AICD after encountering the cognate antigen in an external DR- and caspase-independent manner and that these CTLs can be rescued from AICD by, for example, disruption of the JNK-AIF signaling cascade. In addition, the invention comprises methods for upregulating JNK and/or AIF induce AICD to treat pathologies associated with aberrant T cell activity.

In certain aspects, the present invention relates to methods for improving the treatment of cancer by enhancing the survival of tumor antigen-specific CTLs induced by cancer immunotherapy, for example, active specific vaccination and/or adoptive therapy. The present invention also relates to methods for rescuing CTLs from apoptosis, for example AICD or PCD, through the administration of an active agent, for example, a JNK inhibitor, an inhibitory RNA or combinations thereof

In an embodiment, the invention relates to a method of enhancing the survival of an antigen activated CTL from death comprising administering to a T cell an effective amount of a c-jun N-terminal kinase (JNK) inhibitor, for example, a pyrazoloanthrone or derivative thereof. In another of the embodiments, the invention relates to a method of enhancing the survival of an antigen activated CTL by administering to a T cell an effective amount of anthra[1,9-cd]pyrazol-6(2H)-one.

In certain aspects the method of the invention relates to the administration of an effective amount of a compound having the general formula (I), derivatives, and pharmaceutically acceptable salts and bases thereof, along with pharmaceutically acceptable excipients, for the prevention of T cell death. In the general formula (I), R¹-R³ are optional substituents that are the same or different and independently represent, for example, hydrogen, hydroxyl, alkyl, halogen, nitro, trifluoromethyl, sulfonyl, carboxyl, alkoxycarbonyl, alkoxy, aryl, phenyl, naphthyl, carbocyclic or heterocyclic aromatics, phenyl, furanyl, theinyl, imidazolyl, thiazolyl, pyrazolyl, pyridazinyl, pyrazinyl, triazinyl, tetrazolyl, indolyl, pyrimidinyl, pyridinyl, aryloxy, arylalkyloxy, arylalkyl, cycloalkyl, cycloalkyloxy, alkoxyalkyl, alkoxyalkylalkoxy, aminoalkoxy, arylamino, arylalkylamino, alkylamino, cycloalkylamino, mono- or di-alkylaminoalkoxy, or combinations thereof

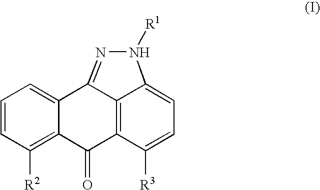

Bennett et al. (2001) has previously shown that the pyrazoloanthrone, anthra[1,9-cd]pyrazol-6(2H)-one, can inhibit c-jun N-terminal kinase (Kᵢ≈190 nM). (2001) PNAS, 98(24):13681-686. In addition, methods of synthesizing pyrazoloanthrones, for example SP600125, derivatives, and pharmaceutically acceptable salts and bases thereof are shown and described in U.S. patent application Ser. No. 10/738,640 to Bennett (Pub. No.: US 2004/0176434 A1). These documents are hereby incorporated by reference in their entirety into the present application.

Another aspect of the present invention relates to providing methods for activating antigen specific T cells cultured ex vivo, for example, MART-1 specific CTLs. In certain aspects of this object, antigen specific T cells, for example CTLs, are isolated from a donor/host organism. In certain embodiments the donor host has cancer, for example melanoma.

A particularly preferred object of the present invention relates to a method for inhibiting apoptosis and increasing the population of ex vivo cultured antigen specific CTLs. In certain aspects this method includes treating antigen specific CTLs activated according to the methods disclosed herein, with an effective amount of a pyrazoloanthrone, or pyrazoloanthrone derivative, for example anthra[1,9-cd]pyrazol-6(2H)-one.

In the process of enhancing the survival of antigen specific CTLs, JNK inhibitors interfere with their capacity to produce IFN-γ but does not interfere with their cytolytic function. Furthermore, the rescued epitope specific CTLs, regain their capacity to synthesize IFN-γ when continued in culture with IL-15 and without the inhibitor. Therefore, it is another object of the present invention to provide a method of treating cancer in a donor/host organism in need thereof, comprising the additional step of administering back to said donor/host the activated antigen specific CTLs that have been propagated ex vivo according to the methods of the present invention.

In another aspect, the present invention features a nucleic acid molecule, such as an enzymatic nucleic acid, for example, a decoy RNA, dsRNA, siRNA, shRNA, micro RNA, aptamers, antisense nucleic acid molecules, which down regulates expression of a sequence encoding a c-jun N-terminal kinase (JNK). The invention also features an enzymatic nucleic acid molecule which down regulates expression of a sequence encoding AIF. In an embodiment, a nucleic acid molecule of the invention is adapted to treat cancer. In another embodiment, an enzymatic nucleic acid molecule of the invention has an endonuclease activity to cleave RNA having JNK or AIF nucleic acid sequence, i.e., SEQ ID NO:1 and 3. The enzymatic nucleic acids contemplated by the invention are specific or complementary for the target gene mRNA. In one embodiment, an enzymatic nucleic acid molecule of the invention is in an Inozyme, Zinzyme, G-cleaver, Amberzyme, DNAzyme, or Hammerhead configuration. In certain embodiments, the invention relates to siRNA expression constructs comprising at least one DNA sequence encoding an siRNA contained in an expression vector operably linked to at least one DNA regulatory element. In certain embodiments, the DNA sequence encoding the siRNA of the invention comprise SEQ ID NOs:5, 6, 7, or combinations thereof. As one of skill in the art will recognize, the nucleic acid that forms the siRNA is present in the expression vector as double stranded DNA and is transcribed to an RNA which is composed of a single strand of ribonucleotides, including uracil instead of thymine. Alternatively, the nucleic acid that forms the siRNA can be present in an RNA viral vector, which is converted to double stranded DNA by a reverse transcriptase and then transcribed to RNA.

In one embodiment, a nucleic acid molecule of the invention comprises between 12 and 100 bases complementary to RNA having a JNK or AIF nucleic acid sequence, i.e., SEQ ID NO: 1 and 3. In another embodiment, a nucleic acid molecule of the invention comprises between 14 and 24 bases complementary to RNA having a nucleic acid sequence having at least 85% homology to SEQ ID NO: 1 and 3.

In yet another embodiment, a nucleic acid molecule of the invention is chemically synthesized.

In another embodiment, a nucleic acid molecule or antisense nucleic acid molecule of the invention comprises at least one 2′-sugar modification, at least one nucleic acid base modification, or at least one phosphate backbone modification.

In one embodiment, a siRNA molecule of the invention comprises a double stranded RNA wherein one strand of the RNA is complementary to the RNA of a JNK or AIF gene. In another embodiment, a siRNA molecule of the invention comprises a double stranded RNA wherein one strand of the RNA comprises a portion of a sequence of RNA having a JNK or AIF gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop, hairpin or stem loop structure.

In one embodiment, a single strand component of a siRNA molecule of the invention is from about 14 to about 56 nucleotides in length. In another embodiment, a single strand component of a siRNA molecule of the invention is from about 14 to about 28 nucleotides in length. In yet another embodiment, a single strand component of a siRNA molecule of the invention is from about 19 to about 25 nucleotides in length.

In another embodiment, an enzymatic nucleic acid molecule, antisense nucleic acid molecule, decoy RNA, dsRNA, siRNA, or aptarner molecules of the invention comprises at least one 2′-sugar modification. In another embodiment, an enzymatic nucleic acid molecule, antisense nucleic acid molecule, decoy RNA, dsRNA, siRNA, or aptamer, nucleic acids of the invention comprises at least one nucleic acid base modification. In another embodiment, an enzymatic nucleic acid molecule, antisense nucleic acid molecule, decoy RNA, dsRNA, siRNA, or aptamer, nucleic acids of the invention comprises at least one phosphate backbone modification.

In one embodiment, the invention features a mammalian cell, for example a human cell, including a nucleic acid molecule of the invention.

The present invention features method of down-regulating JNK and/or AIF expression or activity in a cell, comprising contacting the cell with an enzymatic nucleic acid molecule or antisense nucleic acid molecule, or other nucleic acid molecule of the invention, under conditions suitable for down-regulating of JNK and/or AIF expression or activity.

The present invention also features method of treatment of a subject having a condition associated with the level of JNK, comprising contacting cells of the subject with an enzymatic nucleic acid molecule or antisense nucleic acid molecule or other nucleic acid molecule of the invention under conditions suitable for the treatment.

The present invention also features method of treatment of a subject having a condition associated with the level of AIF expression, comprising contacting cells of the subject with the enzymatic nucleic acid molecule or antisense nucleic acid molecule or other nucleic acid molecule of the invention, under conditions suitable for the treatment.

In one embodiment, a method of treatment of the invention comprises the use of one or more drug therapies under conditions suitable for said treatment.

The present invention features methods of cleaving RNA comprising a JNK nucleic acid sequence comprising contacting an enzymatic nucleic acid molecule of the invention with the RNA under conditions suitable for the cleavage.

The present invention also features methods of cleaving RNA comprising a AIF nucleic acid sequence comprising contacting an enzymatic nucleic acid molecule of the invention with the RNA under conditions suitable for the cleavage.

In one embodiment, a method of cleavage of the invention is carried out in the presence of a divalent cation, for example Mg2+.

In another embodiment, an enzymatic nucleic acid or antisense nucleic acid molecule or other nucleic acid molecule of the invention comprises a cap structure, wherein the cap structure is at the 5′-end, or 3′-end, or both the 5′-end and the 3′-end, for example a 3′, 3′-linked or 5′, 5′-linked deoxyabasic derivative.

The present invention also features an expression vector comprising a nucleic acid sequence encoding at least one enzymatic nucleic acid molecule, antisense, or other nucleic acid molecule of the invention in a manner which allows expression of the nucleic acid molecule.

In one embodiment, the invention features a mammalian cell, for example a human cell, including an expression vector contemplated by the invention.

In another embodiment, an expression vector of the invention further comprises an antisense nucleic acid molecule complementary to RNA of a JNK or AIF.

In yet another embodiment, an expression vector of the invention comprises a nucleic acid sequence encoding two or more enzymatic nucleic acid molecules, which can be the same or different.

The present invention also features a method for treatment of cancer, for example breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer, comprising administering to a subject an enzymatic nucleic acid molecule or antisense nucleic acid molecule or other nucleic acid molecule of the invention under conditions suitable for said treatment.

In one embodiment, a nucleic acid molecule of the invention comprises at least five ribose residues, at least ten 2′-O-methyl modifications, and a 3′-end modification such as a 3′-3′ inverted abasic moiety, and/or phosphorothioate linkages on at least three of the 5′ terminal nucleotides.

In another embodiment, other drug therapies contemplated by the invention include monoclonal antibodies, JNK and/or AIF inhibitors, chemotherapy, or radiation therapy. Specific chemotherapy contemplated by the invention include paclitaxel, docetaxel, cisplatin, methotrexate, cyclophosphamide, 5-fluoro uridine, Leucovorin, Irinotecan (CAMPTOSAR.RTM. or CPT-1 or Camptothecin-11 or Campto), Paclitaxel, Carboplatin doxorubin, fluorouracil carboplatin, edatrexate, gemcitabine, or vinorelbine or a combination thereof.

The invention also features a method for treatment of an inflammatory disease, for example rheumatoid arthritis, restenosis, asthma, Crohn's disease, diabetes, obesity, autoimmune disease, lupus, multiple sclerosis, transplant/graft rejection, gene therapy applications, ischemia/reperfusion injury, glomerulonephritis, sepsis, allergic airway inflammation, inflammatory bowel disease, or infection, comprising the step of administering to a subject an enzymatic nucleic acid or antisense nucleic acid molecule of the invention under conditions suitable for the treatment.

The present invention features compositions comprising the enzymatic nucleic acid and/or antisense nucleic acid molecules of the invention in a pharmaceutically acceptable carrier.

The invention also features a method of administering to a cell, such as mammalian cell (e.g. human cell), where the cell can be in culture or in a mammal, such as a human, an enzymatic nucleic acid molecule or antisense molecule of the instant invention, comprising contacting the cell with the enzymatic nucleic acid molecule or antisense molecule or other nucleic acid molecule of the invention under conditions suitable for such administration. The method of administration can be in the presence of a delivery reagent, for example a lipid, cationic lipid, phospholipid, or liposome.

A further object of the present invention is to provide a kit comprising a suitable container, the active agent of the invention in a pharmaceutically acceptable form disposed therein, and instructions for its use.

Additional objects and advantages of the present invention will be appreciated by one of ordinary skill in the art in light of the current description and examples of the preferred embodiments, and are expressly included within the scope of the present invention.

### BRIEF DESCRIPTION OF THE DRAWINGS

FIG. 1. AICD in MART-1₂₇₋₃₅ epitope specific human primary CTL is blocked by JNK inhibitor SP600125 and does not involve caspase-8 and caspase-3 activation. (A) Effect of the JNK inhibitor SP600125 on AICD in the MART-1₂₇₋₃₅ epitope specific CTL. Histogram represents FACS analysis of the percent of MART-1₂₇₋₃₅ epitope specific CTLs that were tetramer positive and annexin negative after restimulation with MAGE-3₂₇₁₋₂₇₉ control peptide (M3) pulsed T2 cells or MART-1₂₇₋₃₅ peptide (M1) pulsed T2 cells either alone or re-stimulated after pre-incubation with JNK inhibitor SP600125, p38-kinase inhibitor SB203580, ERK1/2 inhibitor PD98059 or caspase-inhibitor z-VAD-fink. MAGE-3₂₇₁₋₂₇₉. (B) JNK activation results in phosphorylation of c-Jun during AICD in CTL that is blocked by SP600125. Immunoblots show analyses of CTL extracts probed with anti-phosphor-c-Jun, anti-c-Jun, and anti-actin antibodies. C-control CTL, M1-CTL re-stimulated with the cognate peptide Mart-1_{27-35,} M3-CTL re-stimulated with control peptide, Mage-3₂₇₁₋₂₇₉, and SP-CTL re-stimulated with Mart-1₂₇₋₃₅ peptide after pre-incubation with the JNK inhibitor, SP600125. (C) Caspase-8 and caspase-3 activation assay in the MART-1₂₇₋₃₅ epitope specific CTL. Immunoblots show analyses of CTL extracts probed with anti-caspase-8, anti-caspase-3 and anti-actin antibodies. Panel (i)—C: CTL control; M1: re-stimulated with MART-1₂₇₋₃₅ peptide; M3: re-stimulated with MAGE-3₂₇₁₋₂₇₉ control peptide); panel (ii) shows a positive control for caspase-8 and caspase-3 following 1 μM Staurosporine (Stau) treatment for 4 hr. (D) Blocade of JNK activation and PARP cleavage by SP600125 and z-VAD-fmk. (i) Jurkat cells were treated with PMA+ionomycin in the absence or presence of SP600125 and JNK activation (c-Jun phosphorylation) was assessed with anti-phospho-c-Jun antibody. Antibodies to c-Jun and actin were used to measure total c-Jun protein and actin served as a loading control. (ii) Jurkat cells were treated with Staurosporine for 1 and 4 hrs in the absence (S) or presence of caspase inhibitor, z-VAD-fmk (CI), JNK inhibitor, SP600125 (SP) or ERK1/2 inhibitor PD98059 (PD) and cleavage of PARP was detected by immunoblotting, using actin as a loading control. Untreated Jurkat cells were used as control (C). Percent annexin positive cells in all samples were quantified by FACS analysis and shown below the blot

FIG. 2. Evidence of single stranded DNA nicks and large-scale DNA fragmentation in AICD of the MART-1₂₇₋₃₅ epitope specific primary CTL. (A) Large scale DNA fragmentation in the CTL during AICD. Standard 2% agarose gel-electrophoresis (panel i) and pulse field DNA gel electrophoresis (panel ii) of: CTL in steady state (C) and after restimulation by the cognate peptide (M1). Panel (iii) shows standard 2% agarose gel electrophoresis of DNA the CTL in steady state (C) and after the treatment with 1 μM staurosporine for 4 hr (Stau). (B) Flow-cytometric quantitation of ssDNA nick positive (i) and PI positive (ii): (a), epitope specific CTL in steady state; (b), CTL exposed to T2 cells, pulsed with the control peptide MAGE-3₂₇₁₋₂₇₉; (c), CTL exposed to T2 cells, pulsed with the cognate peptide MART-1₂₇₋₃₅.

FIG. 3. Release of apoptotic proteins from mitochondria into cytosolic fraction of primary CTL during AICD. Immuno-blot analysis of heavy membrane (HM) and cytosolic fractionations (S-100) of MART-1₂₇₋₃₅ CTL re-stimulated with control peptide (C) and re-stimulated with MART-1₂₇₋₃₅ peptide (M1).

FIG. 4. Schematic diagram of recombinant lenti viruses encoding short hairpin (Sh) RNA targeting JNK and AIF genes. The lenti virus construct comprises, a loop or small hairpin flanked on both sides by self-hybridizing DNA sequences (i.e., sense and antisense (i.e., the reverse complement of the sense sequence)) specific to the gene of interest, for example SEQ ID NOs: 5-7; a 3′ self inactivating long terminal repeat (SIN/LTR); a central polypurine tract (cppt); a RNA packaging signal (Psi); a cytomegalovirus immediate-early promoter (CMV) upstream of a reporter gene, in this case green fluorescent protein; and a posttranscriptional regulatory element.

FIG. 5. Analyses of JNK and AIF knockdown cell lines. (A). Representative FACS analysis of 293 (A) cell line for GFP positivity. (B). Western blot analysis of 293 JNK and AIF knowdown cell lines.

FIG. 6. (A). FACS analysis of Jurkat (B) cell line for GFP positivity. (B). Western blot analysis of Jurkat JNK and AIF knockdown cell lines.

FIG. 7. Effect of JNK and AIF knockdown on TNF- alpha (A) and Fas (B) mediated cell death of Jurkat cells. [Control-Negative Control; Control Lenti-Jurkat cells with integrated control Lenti virus exposed to 10 ng TNF-alpha (A) or Fas (B); JNK2-clone2-Jurkat cells with integrated Lenti virus encoding JNK siRNA, exposed to 10 ng TNF-alpha (A) or Fas (B); AIF-Jurkat cells with integrated Lenti virus encoding AIF siRNA, exposed to 10 ng TNF-alpha (A) or Fas (B)].

FIG. 8. An example of activation and expansion of MART-1₂₇₋₃₅ epitope specific CTLs in the in vitro CTL generation culture. (A) Number of HLA-A2/MART 1₂₇₋₃₅ tetramer positive cells from a normal donor [top], and from a melanoma patient [bottom]. (B) IFN-γ response by the corresponding population (M3=MAGE-3₂₇₁₋₂₇₉, M1=MART-1₂₇₋₃₅).

FIG. 9. Effector function and the fate of MART-1₂₇₋₃₅ epitope specific CTLs upon secondary encounter of the cognate epitope. (A) IFN-γ response by the effector cells. (M3=MAGE-3₂₇₁₋₂₇₉, M1=MART-1₂₇₋₃₁) (B) Evidence of early death in the antigen specific effector cells after secondary encounter of the epitope. Top panel=Number of MART-1₂₇₋₃₅ tetramer⁺ CD8⁺ T cells; Bottom panel=Mart-1₂₇₋₃₅ tetramer⁺/annexin V⁺ populations. The numbers at the top of the bottom panel represent the mean +S.E. of three replicate samples while “a,” “b,” “c,” and “d” in the superscript indicate that an increase in the MART-1₃₇₋₃₅ tetramer⁺/annexin V⁺ cells in “a,” when compared to that in “b,” “c,” and “d,” was significant with p<0.001 (One way ANOVA). (C) Survival of the MART-1₂₇₋₃₅ specific population after secondary encounter of the epitope. The effector cells were co-incubated with peptide (1 μg/ml) pulsed irradiated T cells (CTL: T2=100). The cultures were washed about 18 h later and was then maintained in IL-15 containing medium for about 5 days. The recovery of the antigen specific CTLs was determined by counting the number of viable cells (trypan blue negative population) and by factoring in the number of antigen specific population (% tetramer⁺ population). The difference in the % tetramer⁺ CTLs rescued after five days post secondary stimulation with MART-1 peptide loaded T2 cells, when compared to other groups was significant at *p≦0.001 (One way ANOVA). FIG. 9A and B represent one of 5 separate experiments. FIG. 9C represents one of three separate experiments.

FIG. 10. Effect of external death receptor blockade and MAP kinase inhibitors on AICD in the epitope specific CTLs upon secondary encounter of the cognate epitope. The CTLs were pre-incubated at optimal concentration for about 45 min. at about 37° C. with pan caspase inhibitor z-VAD-fmk (100 μM); human Fas/Fc chimera, human TNF-RI/Fc chimera, human TRAIL-RI/Fc chimera, human TRAIL-RII/Fc chimera and human IFN-γ RI/Fc chimeric proteins (10 μg/ml); p38 kinase inhibitor SB203580, JNK inhibitor anthra[1,9-cd]pyrazol-6(2H)-one (SP600125), JNK/Erk inhibitor PD98059 (25 μM). Then the pretreated as well as untreated CTLs were incubated with T2 cells either alone or loaded with peptide. About 4 hours after secondary exposure cells were stained for determining: (A) Number of tetramer⁺ cells out of the CD8⁺ T cells [Top panel]; number of tetramer⁺/Annexin V⁺ and tetramer⁺/Annexin⁻ populations [Bottom panel]. The numbers on the top margin of the bottom panel represent tetramer⁺/Annexin⁻ from the mean±SE of three replicate samples while “a,” “b,” “c,” and “d” in the superscript indicate increase in the number of tetramer⁺/annexin V⁻ population in “a,” when compared to that in “b,” “c,” and “d” was significant with p<0.001 (One way ANOVA). (B) Effect of the MAP kinase inhibitors on IFN-γ response by the effector cells. (M3=MAGE-3₂₇₁₋₂₇₉, M1=MART-1₂₇₋₃₅. The reduction in IFN-γ synthesis in a, when compared to b, c and d was significant at p≦0.001 by one way ANOVA. M3=MAGE-3₂₇₁₋₂₇₉, M1=MART-1₂₇₋₃₅. (C) Effect of the MAP kinase inhibitor on cytotoxic response by the effector cells. (M3=MAGE-3₂₇₁₋₂₇₉, M1=MART-1₂₇₋₃₅). The difference in the % specific lysis in the MART-1 peptide loaded T2 cells was significant (*p≦0.001 by Student's t-test) only when compared to that of MAGE-3₂₇₁₋₂₇₉ peptide loaded T2 cells at all effector to target ratios (E:T). FIG. 10A represents one of four separate experiments while FIG. 10B and 10C represent one of two separate experiments.

FIG. 11. Evidence for a caspase independent pathway for apoptosis in Mart-1 specific primary CTLs. (A) The CTLs were pre-incubated at optimal concentration for about 45 min. at 37° C. with p38 kinase inhibitor SB203580 and JNK inhibitor anthra[1,9-cd]pyrazol-6(2H)-one (SP600125) (25 μM). The pretreated as well as untreated CTLs were then incubated with T2 cells either alone or loaded with peptide. About 6 hours after secondary exposure cells were used for western blot analysis for PARP cleavage [top panel]. Jurkat T cells either pre-incubated with pan caspase inhibitor z-VAD-fmk (100 μM) or untreated were exposed with staurosporine (1 μM) to establish the PARP cleavage alongside. (B) Jurkat T cells were stimulated with PMA (10 ng/ml) /ionomycin (0.5 μM) for indicated time both in the presence and absence of JNK inhibitor SP600125 (25 μM). Immunoprecipitation was done to ascertain for the blockade of c-jun phosphorylation/activation in presence of SP600125, as seen in the blot.

FIG. 12. Effect of antigen specific secondary stimulation on mitochondrial transmembrane membrane potential (Δψₘ), pro- and anti-apoptotic protein and expression of cell surface molecules. (A) Differential effect on membrane polarization in Mart-1 primary CTLs (top panel) vs. Jurkat T cells (bottom panel). CTLs were incubated with T2 cells either alone or loaded with peptide. After overnight secondary exposure cells were stained with Mart-1 tetramer/CD8 and DiOC₆. Histogram represents the fluorescence of DiOC₆ in the gated antigen specific tetramer⁺ population (top panel). Jurkat T cells were also analyzed for modulation of membrane potential after TCR-driven stimulation (bottom panel). Values represent the mean±SD of fluorescence intensity triplicates from one out of three separate experiment. *p<0.001 (One Way ANOVA). (B) Left panel shows western blots for anti- and pro-apoptotic proteins using the whole CTL population following secondary stimulation in the presence or absence of the MAP kinase inhibitors. Right panel, shows the densitometric analysis of the relevant Bcl family proteins normalized to β-actin controls using OPTIMAS Version 5.2 software (Bioscan, Bothell, Wash.). (C) Expression of different cell surface receptors for survival or death signal in antigen specific population upon secondary stimulation. Values on left upper corner represent the geometric mean of the fluorescence intensity for that particular marker when stimulated with T2/MAGE-3₂₇₁₋₂₇₉ while that on right upper corner represent the geometric mean of the fluorescence intensity when stimulated with T2/MART-1₂₇₋₃₅.

FIG. 13. Survival of the antigen specific CTLs following secondary encounter of the epitope in the presence of anthra[1,9-cd]pyrazol-6(2H)-one (SP600125) and recovery of function in the pyrazoloanthrone rescued CTLs. Effector cells were co-cultured with peptide pulsed T2 cells in the presence or absence of the kinase inhibitors (25 μM). The cultures were washed about 18 h later and then recultured in IL-15 containing medium. On day five, the numbers of the viable antigen specific CTLs were determined. (A) Top panel shows the forward and side scattering of the total population on day five and the number of viable cells that could be recovered and gated for analysis. The number (11%) of MART-1₂₇₋₃₅ tetramer⁺/CD8⁺ population exposed to MART-1₂₇₋₃₅ peptide pulsed T2 cells (T2/M1) is a reflection of loss of epitope specific CTLs from AICD. Bottom panel shows the percentage of MART-1₂₇₋₃₅ tetramer⁺/CD8⁺ cells recovered five days after co-culture of the CTLs with peptide pulsed T2 cells in the presence or absence of SP600125. (B) IFN-γ response by the recovered populations (M3=MAGE-3₂₇₁₋₂₇₉, M1=MART-1₂₇₋₃₅).

FIG. 14. Nuclear translocation of AIF and effect of SP600125 on the AIF release and such translocation. The MART-1 epitope specific CTLs generated in the in vitro CTL generation protocol were restimulated by the cognate epitope or a control epitope in the presence or absence of the JNK inhibitor SP600125. (A) Western blot of heavy membrane faction (HM) and cytosolic fraction (S-100) of control CTL (C) and CTLs restimulated by the cognate peptide (M1). As shown, AIF is released in the cytosol of CTLs encountering the cognate peptide without any release of cytochrome c. (B) Effect of SP600125 on AIF release in CD3 induced AICD in Jurkat cells. As shown, AIF is released without cytochrome c release in these cells and SP600125 interferes with the AIF release. (C) Densitometric analysis of the SP600125 effect on AIF release.

FIG. 15. AIF-mediated DNA fragmentation in the MART-1 specific CTLs during AICD. (A)—Flowcytometric quantitation of ssDNA fragmentation at different time points. (Panel i), Flowcytometric analyses of ssDNA nicked cells (a, control cells; b, CTLs encountering a control peptide; c, CTLs encountering the cognate peptide. (Panel ii) represents P I staining showing increase in the number of dead cells with the CTLs encountering the cognate peptide. (B)—Agarose gel analysis of DNA during AICD. (i)=Regular gel (1% agarose) showing no oligosomal DNA fragmentation (c, control DNA and M1=DNA from CTL undergoing AICD; (ii), Pulse field gel electrophoresis of control DNA (C) and DNA during AICD (M1) showing larger fragmentation (around 50 kbp) during AICD; (iii)=Regular gel electrophoresis of DNA from the control CTLs (C) and from CTLs undergoing apoptosis showing oligosomal DNA fragmentation upon Staurosporine treatment).

FIG. 16. Mitochondrial localization of JNK in the CTL. (A)—Heavy membrane fractions from CTLs in steady state (C), restimulated by the cognate peptide (M1) and control peptide (M3) and the corresponding cytosolic fractions (S-100) were probed for the presence for JNKs. As shown, JNK preferentially localized in the membrane fraction. (B)—The generation of short pBim fragment during AICD and the effect of SP600125 on the generation of this fragment. Mart-1 epitope specific control CTLs (c), and CTLs encountering the cognate peptide (M1), or encountering a control peptide (M3) and encountering the cognate peptide in the presence of SP600125 (SP) or encountering the cognate peptide in the presence of ERK1/2 inhibitor (PD)were probed with an anti-phosphorylated Bim antibody. A phosphorylated short fragment of Bim is generated in the CTLs encountering the MART-1 peptide and this is blocked by SP600125. (C)—Western blot of whole cell lysate of CTLs showing interaction between JNK and VDAC in CTLs (c=control, M1=CTL restimulated by the cognate peptide) Anti-CD8 Ab was used as a control antibody for immunoprecipitation.

### DETAILED DESCRIPTION OF THE INVENTION

In the present invention, we use the Melan-A/MART-1₂₇₋₃₅ epitope as a prototype example of a self-but-melanoma associated antigen for demonstrating the inhibition/prevention of AICD in epitope specific primary CTLs. However, the invention is not limited in this regard and, as one of ordinary skill in the art will recognize, any antigen specific CTL could be used in the methods of the present invention and all such CTLs are expressly included within the scope of the present specification and claims.

In an embodiment the invention comprises a method for rescuing antigen specific CTLs from apoptosis, for example AICD or PCD, through the administration of an effective amount of an active agent, for example a JNK inhibitor, an inhibitory RNA, and combinations thereof, and optionally including at least one other therapeutic agent and/or pharmaceutically acceptable excipients or carriers. In a preferred embodiment, the active agent comprises a pyrazoloanthrone or derivatives and pharmaceutically acceptable salts and bases thereof, for example, the c-jun N-terminal kinase (JNK) inhibitor, anthra[1,9-cd]pyrazol-6(2H)-one, commercially available as SP600125 (Biomol International, PA).

In certain aspects the method of in the invention relates to the administration of an effective amount of a pyrazoloanthrone having the general formula (I), derivatives, and pharmaceutically acceptable salts and bases thereof, along with pharmaceutically acceptable excipients and/or carriers, for the prevention of T cell death. In the general formula (I), R¹-R³ are optional substituents that are the same or different and independently represent, for example, hydrogen, hydroxyl, alkyl, halogen, nitro, trifluoromethyl, sulfonyl, carboxyl, alkoxycarbonyl, alkoxy, aryl, phenyl, naphthyl, carbocyclic or heterocyclic aromatics, phenyl, furanyl, theinyl, imidazolyl, thiazolyl, pyrazolyl, pyridazinyl, pyrazinyl, triazinyl, tetrazolyl, indolyl, pyrimidinyl, pyridinyl, aryloxy, arylalkyloxy, arylalkyl, cycloalkyl, cycloalkyloxy, alkoxyalkyl, alkoxyalkylalkoxy, aminoalkoxy, arylamino, arylalkylamino, alkylamino, cycloalkylamino, mono- or di-alkylaminoalkoxy, or combinations thereof, along with pharmaceutically acceptable excipients, to CTLs in vivo, cultured ex vivo, or both for the prevention/inhibition of T cell death.

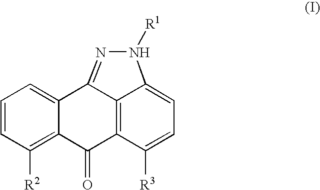

Another of the preferred embodiments comprises methods for generating activated antigen specific T cells, for example, MART-1 specific CTLs, cultured ex vivo. In certain aspects of this embodiment, antigen specific T cells, for example CTLs, are isolated from a donor/host organism. In certain embodiments the donor host has cancer, for example melanoma. In an important aspect of this object, monocytes, immature dendritic cells (DCs), or peripheral blood mononuclear cells (PBMCs) are isolated from the same host organism. The immature DCs are stimulated to mature by incubation for about 2 hours in the presence of IFN-γ (about 1000 U/ml), and lipopolysaccharide (LPS) (about 100 ng/ml). In a particular aspect of this object, the mature DCs are incubated with antigen specific CTLs at a ratio of about 1 to from about 1 to 1000. In a preferred embodiment the ratio of DCs to CTLs is about 1 to from about 50 to about 500. In a particularly preferred embodiment the ratio of DCs to CTLs is 1:100. In certain aspects the incubation mixture contains DCs, CTLs, and IL-15 at a concentration of from about 0.1 ng/ml to about 100 ng/ml. In a preferred embodiment the concentration of IL-15 used is from about 1 ng/ml to about 50 ng/ml.

A particularly preferred embodiment of the present invention includes a method for inhibiting apoptosis and increasing the population of ex vivo cultured antigen specific CTLs. Certain aspects of this embodiment include treating antigen specific CTLs activated according to the methods disclosed herein, with an effective amount of a pyrazoloanthrone, or pyrazoloanthrone derivative, for example anthra[1,9-cd]pyrazol-6(2H)-one, at a concentration of from about 1 μM to about 1 mM. In certain of the preferred embodiments the concentration of the pyrazoloanthrone is from about 10 μM to about 100 μM. In a preferred embodiment the pyrozoloanthrone is incubated in the presence of the activated antigen specific CTLs for from about 10 minutes to about 24 hours. In another of the preferred embodiments the pyrazoloanthrone is incubated with activated antigen specific CTLs for from about 30 minutes to about 1 hour.

In another embodiment, the invention comprises CTLs generated according to any of the methods of the invention. In any of the methods disclosed herein the DC and CTLs cells are incubated at a temperature of from about 20° C. to about 40° C. In a particularly preferred embodiment cell culture incubations are performed at a temperature of about 37° C.

In another of the preferred embodiments the present invention includes a method of treating cancer in a donor/host organism in need thereof, comprising isolating from the host a population of T cells from a host, generating activated antigen specific CTLs that have been propagated ex vivo according to the methods of any of the preferred embodiments, administering to the CTL cells an effective amount of a JNK inhibitor, and administering back to said donor/host the activated and propagated antigen specific CTLs. The present invention also includes methods for treating cancer comprising generating activated antigen-specific CTLs in vivo by active specific vaccination, and administration of an effective amount of a JNK inhibitor.

Still another of the preferred embodiments includes a method of inhibiting/preventing apoptosis of CTLs in vivo for the treatment of cancer comprising the administration of an anticancer agent in combination with the simultaneous or subsequent administration of an effective amount of a JNK inhibitor, for example, a pyrazoloanthrone, an anthra[1,9-cd]pyrazol-6(2H)-one, or derivates thereof, to an organism in need thereof. Certain aspects of this embodiment include the administration of a pyrazoloanthrone, for example anthra[1,9-cd]pyrazol-6(2H)-one, in combination with at least one other therapeutic agent, for example, an analgesic, an antibiotic, an anti-inflammatory, an NSAID, an opioid, an antineoplastic, for example, paclitaxel, docetaxel, cisplatin, methotrexate, cyclophosphamide, 5-fluoro uridine, Leucovorin, Irinotecan (CAMPTOSAR.RTM. or CPT-11 or Camptothecin-11 or Campto), Paclitaxel, Carboplatin doxorubin, fluorouracil carboplatin, edatrexate, gemcitabine, or vinorelbine, alkeran, melphalan, mustargen, mechlorethamine, carmustine, oxaliplatin, vinblastine, idarubicin, loracarbef, epirubicin, chlorambucil, warfarin, allopurinol, and combinations thereof. Further aspects of this embodiment include the administration of the pyrazoloanthrone sequentially, as distinct from simultaneously, with the administration of at least one other therapeutic agent.

Another of the preferred embodiments includes a method for inhibiting apoptosis in a cell, for example a CTL cell, comprising the administration of an effective amount of an active agent capable of inhibiting the release of apoptosis inducing factor (AIF) from the mitochondria of a cell. In certain aspects of this embodiment of the invention, the active agent is a pyrazoloanthrone or pyrazoloanthrone derivative. In a related embodiment, the present invention includes a method for inducing apoptosis in a T cell by administration of an active agent that induces the release of AIF from the mitochondria of a cell and translocation to the nucleus.

The invention also features nucleic acid molecules, for example enzymatic nucleic acid molecules: antisense nucleic acid molecules, 2,5-A chimeras, decoys, double stranded RNAs, small inhibitory RNAs, micro RNAs, short loop or hairpin RNAs, triplex oligonucleotides, and/or aptamers, and methods to modulate gene expression, for example, expression of genes encoding a JNK and/or AIF protein (See SEQ ID NOs: 1-4). In particular, the instant invention features nucleic-acid based molecules and methods to modulate the expression of a JNK kinase and/or an AIF protein.

The invention features one or more enzymatic nucleic acid-based molecules and methods that independently or in combination modulate the expression of gene(s) encoding a JNK kinase (SEQ ID NO:1; Genbank Accession No. NM_{—}139049) and/or an AIF protein (SEQ ID NO:3; Genbank Accession NO. AF100928).

The description below of the various aspects and embodiments is provided with reference to the exemplary JNK and AIF genes. However, the various aspects and embodiments are also directed to genes which encode homologs, orthologs, and paralogs of other JNK and AIF genes and include all isoforms, splice variants, and polymorphisms. Those additional genes can be analyzed for target sites using the methods described for JNK and AIF. Thus, the inhibition and the effects of such inhibition of the other genes can be performed as described herein.

By “inhibit” or “down-regulate” it is meant that the expression of the gene, or level of RNAs or equivalent RNAs encoding one or more proteins, or activity of one or more proteins, such as JNK and/or AIF, is reduced below that observed in the absence of the nucleic acid molecules of the invention. In one embodiment, inhibition or down-regulation with enzymatic nucleic acid molecules preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of JNK and/or AIF with the nucleic acid molecule of the instant invention is greater in the presence of the nucleic acid molecule than in its absence.

By “up-regulate” is meant that the expression of the gene, or level of RNAs or equivalent RNAs encoding one or more proteins, or activity of one or more proteins, such as JNK and/or AIF, is greater than that observed in the absence of the nucleic acid molecules of the invention. For example, the expression of a gene, such as JNK (SEQ ID NO:1) and/or AIF (SEQ ID NO:3), can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more proteins, or activity of one or more proteins is up-regulated or down-regulated, such that the expression, level, or activity is greater than or less than that observed in the absence of the nucleic acid molecules of the invention.

By “enzymatic nucleic acid molecule” it is meant a nucleic acid molecule which has complementarity to a specified gene target, and also has or mediates an enzymatic activity resulting in the specific cleavage of a target RNA. That is, the enzymatic nucleic acid molecule is able to intermolecularly cleave RNA, alone or as a component of an enzymatic complex, and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25 31). The nucleic acids can be modified at the base, sugar, and/or phosphate groups. The term “enzymatic nucleic acid” is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, siRNA, micro RNA, short hairpin RNA, endoribonuclease, RNA-induced silencing complexes, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific binding site which is complementary to one or more of the target nucleic acid regions, and that it have nucleotide sequences within or surrounding that binding site which impart a nucleic acid cleaving and/or ligation activity to the molecule (Cech et al., U.S. Pat. No. 4,987,071; Cech et al., 1988, 260 JAMA 3030).

It will be appreciated by one of skill in the art that as referred to herein, SEQ ID NOs: 1, 3, and 5-7 are representative of the DNA sequence, the corresponding RNA sequence (single stranded ribonucleotides containing uracil instead of thymine), and cognate complementary sequences. For example, the phrase “nucleic acid of SEQ ID NO:5” is used in reference to the DNA sequence but also to the RNA and siRNA formed therefrom.

Several varieties of enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA either alone or as part of a multi-protein nuclease complex known as the RNA-induced silencing complex (RISC). Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

By “nucleic acid molecule” as used herein is meant a molecule having nucleotides. The nucleic acid can be single, double, or multiple stranded and can comprise modified or unmnodified nucleotides or non-nucleotides or various mixtures and combinations thereof

By “enzymatic portion” or “catalytic domain” is meant that portion/region of the enzymatic nucleic acid molecule essential for cleavage of a nucleic acid substrate.

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092 2096; Hammann et al., 1999, Antisense and Nucleic Acid Drug Dev., 9, 25-31). That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to three nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herranz et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” or “related” RNA to JNK or AIF is meant to include those naturally occurring RNA molecules having homology (partial or complete) to JNK or AIF proteins or encoding for proteins with similar function as JNK or AIF proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

Long double-stranded RNAs (dsRNAs; typically >200 nt) can be used to silence the expression of target genes in a variety of organisms and cell types (e.g., worms, fruit flies, plants, and mammals). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response. exemplified by nonspecific inhibition of protein synthesis and RNA degradation. The mammalian antiviral response can be bypassed, however, by the introduction or expression of siRNAs.

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bernards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

Some vectors have been engineered to express small hairpin RNAs (shRNAs), which get processed in vivo into siRNAs-like molecules capable of carrying out gene-specific silencing. The vectors contain the shRNA sequence between a polymerase III (pol III) promoter (e.g., U6 or H1 promoters) and a 4-5 thymidine transcription termination site. The transcript is terminated at position 2 of the termination site (pol III transcripts naturally lack poly(A) tails) and then folds into a stem-loop structure with 3′ UU-overhangs. The ends of the shRNAs are processed in vivo, converting the shRNAs into ˜21 nt siRNA-like molecules, which in turn initiate RNAi. This latter finding correlates with recent experiments in C. elegans, Drosophila, plants and Trypanosomes, where RNAi has been induced by an RNA molecule that folds into a stem-loop structure. The use of siRNA vectors and expression systems is known and are commercially available from Ambion, Inc.® (Austin, Tex.), Lentigen, Inc. (Baltimore, Md.), Panomics (Fremont, Calif.), and Sigma-Aldrich (ST. Louis, Mo.).

The use of specially designed vector constructs for inducing RNA interference has numerous advantages over oligonucleotide-based techniques. The most significant advantage is stability. Promoter regions in the vector ensure that shRNA transcripts are constantly expressed, maintaining cellular levels at all times. Thus, the duration of the effect is not as transient as with injected RNA, which usually lasts no longer than a few days. And by using expression constructs instead of oligo injection, it is possible to perform multi-generational studies of gene knockdown because the vector can become a permanent fixture in the cell line.

The shRNA templates are constructed as fold-back stem-loop structures that are processed in vivo into siRNA-like transcripts. The stem loop protects the RNA from 3′-5′exonuclease attacks. The coding sequence was inserted beteween restriction sites in a multicloning site. After being processed in the cell, the vector-expressed siRNA is incorporated into a multi-protein nuclease complex known as the RNA-induced silencing complex (RISC). Homology between the sense portion of the siRNA sequence and the mRNA enables the nuclease enzyme to bind and cleave the JNK or AIF encoding transcript into small pieces, which are degraded by the cell's machinery. Once the cleavage of one mRNA molecule has occurred, the RISC is freed from the transcript to perform additional rounds of catalysis.

By “RNase H activating region” is meant a region (generally greater than or equal to 4 25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A chimera” is meant an oligonucleotide, for example an antisense nucleic acid molecule or enzymatic nucleic acid molecule, containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” or “triplex oligonucleotide” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein refers to a double stranded RNA molecule capable of RNA interference “RNAi”, including short interfering RNA “siRNA”, such as short hairpin RNA “shRNA”, see for example Bass, 2001, Nature, 411, 428-429; Elbashir et al., 2001, Nature, 411, 494 498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

By “gene” it is meant a nucleic acid that encodes RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a D-ribo-furanose moiety.

The enzymatic nucleic acid molecule, antisense nucleic acid or other nucleic acid molecules of the invention that down regulate JNK or AIF gene expression represent a therapeutic approach to treat a variety of inflammatory-related diseases and conditions, including but not limited to rheumatoid arthritis, restenosis, asthma, Crohn's disease, incontinentia pigmenti, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other inflammatory disease or condition which respond to the modulation of JNK or AIF function.

The enzymatic nucleic acid molecule, antisense nucleic acid or other nucleic acid molecules of the invention that down regulate JNK or AIF gene expression also represent a therapeutic approach to treat a variety of cancers, including but not limited to skin, breast, lung, prostate, colorectal, brain, esophageal, bladder, pancreatic, cervical, head and neck, and ovarian cancer, melanoma, lymphoma, glioma, multidrug resistant cancers, and/or other cancers which respond to the modulation of JNK or AIF function.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Inhibitory RNA molecules and techniques are described in detail in U.S. Pat. No. 7,022,828, the teachings of which are incorporated herein by reference in their entirety for all purposes.

In one embodiment of the present invention, a nucleic acid molecule of the instant invention can be between about 10 and 100 nucleotides in length. For example, enzymatic nucleic acid molecules of the invention are preferably between about 15 and 50 nucleotides in length, more preferably between about 25 and 40 nucleotides in length (for example see Jarvis et al., 1996, J. Biol. Chem., 271, 29107-29112). Exemplary antisense molecules of the invention are preferably between about 15 and 75 nucleotides in length, more preferably between about 20 and 35 nucleotides in length (see for example Woolf et al., 1992, PNAS, 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length (see for example Maher et al, 1990, Biochemistry, 29, 8820-8826; Strobel and Dervan, 1990, Science, 249, 73-75). Those skilled in the art will recognize that all that is required is that the nucleic acid molecule be of sufficient length and suitable conformation for the nucleic acid molecule to interact with its target and/or catalyze a reaction contemplated herein. The length of the nucleic acid molecules of the instant invention are not limiting within the general limits stated.

Preferably, a nucleic acid molecule that modulates, for example, down-regulates JNK or AIF expression comprises between 12 and 100 bases complementary to a RNA molecule of JNK or AIF. Even more preferably, a nucleic acid molecule that modulates, for example JNK or AIF expression comprises between 14 and 24 bases complementary to a RNA molecule of JNK or AIF.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding JNK or AIF (specifically JNK or AIF genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism. The cell can, for example, be in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “JNK proteins” is meant, a peptide or protein comprising a full length JNK protein, a JNK domain or JNK fragment. By “AIF proteins” is meant, a peptide or protein comprising a full length AIF protein, an AIF domain or AIF fragment.

By “highly conserved sequence region” is meant, a nucleotide sequence of one or more regions in a target gene does not vary significantly from one generation to the other or from one biological system to the other.

Nucleic acid-based inhibitors of JNK or AIF function are useful for the prevention and/or treatment of cancers and cancerous conditions such as skin, breast, lung, prostate, colorectal, brain, esophageal, bladder, pancreatic, cervical, head and neck, and ovarian cancer, melanoma, lymphoma, glioma, multidrug resistant cancers, and any other diseases or conditions that are related to or will respond to the levels of JNK or AIF in a cell or tissue, alone or in combination with other therapies.

Nucleic acid-based inhibitors of JNK or AIF function are also useful for the prevention and/or treatment of inflammatory related diseases and conditions, including but not limited to rheumatoid arthritis, restenosis, asthma, Crohn's disease, incontinentia pigmenti, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other inflammatory disease or condition which respond to the modulation of JNK or AIF function.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers.

In another embodiment, the invention features antisense nucleic acid molecules and 2-5A chimera. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

In another embodiment, the invention features an enzymatic nucleic acid molecule having one or more non-nucleotide moieties, and having enzymatic activity to cleave an RNA or DNA molecule.

In another aspect of the invention, enzymatic nucleic acid molecules or antisense molecules that interact with target RNA molecules and down-regulate JNK or AIF (specifically JNK or AIF gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, lenti virus, cytomegalovirus, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells explanted from the patient or subject followed by reintroduction into the patient or subject, or by any other means that would allow for introduction into the desired target cell. Antisense DNA can be expressed via the use of a single stranded DNA intracellular expression vector.

By “vectors” is meant any nucleic acid-based technique used to deliver a desired nucleic acid, for example, bacterial plasmid, viral nucleic acid, artificial chromosome (e.g., HAC, BAC, and the like).

By “subject” is meant an organism, which is a donor or recipient of explanted cells or the cells themselves. “subject” also refers to an organism to which the nucleic acid molecules of the invention can be administered. Preferably, a subject is a mammal or mammalian cells. More preferably, a subject is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzymes. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, the subject can be treated, or other appropriate cells can be treated, as is evident to those skilled in the art, individually or in combination with one or more drugs under conditions suitable for the treatment.

In a further embodiment, the described nucleic acid molecules, such as antisense or ribozymes, can be used in combination with other known treatments to treat conditions or diseases discussed above. For example, the described molecules can be used in combination with one or more known therapeutic agents to treat cancers of the skin, breast, lung, prostate, colorectal, brain, esophageal, bladder, pancreatic, cervical, head and neck, and ovarian cancer, melanoma, lymphoma, glioma, multidrug resistant cancers, rheumatoid arthritis, restenosis, asthma, Crohn's disease, diabetes, incontinentia pigmenti, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other cancerous disease or inflammatory disease or condition which respond to the modulation of JNK or AIF expression.

In another embodiment, the invention features nucleic acid-based inhibitors (e.g., enzymatic nucleic acid molecules (eg; ribozymes), antisense nucleic acids, 2-5A antisense chimeras, triplex DNA, dsRNA, antisense nucleic acids containing RNA cleaving chemical groups) and methods for their use to down regulate or inhibit the expression of genes (e.g., JNK or AIF) capable of progression and/or maintenance of cancer, inflammatory diseases, and/or other disease states which respond to the modulation of JNK or AIF expression.

Other features and advantages of the invention will be apparent from the following description of the methods and compositions of the invention.

Antisense: Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151 - 190).

In addition, binding of single stranded DNA to RNA can result in nuclease degradation of the heteroduplex (Wu-Pong, supra; Crooke, supra). To date, the only backbone modified DNA chemistry which acts as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Enzymatic Nucleic Acid: Several varieties of enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakacane & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent JNK or AIF protein expression and can be used to treat disease or diagnose disease associated with the levels or activity of JNK or AIF, for example, cancers and immunological diseases.

The enzymatic nature of an enzymatic nucleic acid molecule can allow the concentration of enzymatic nucleic acid molecule necessary to affect a therapeutic treatment to be lower. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to greatly attenuate the catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro. Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate JNK or AIF expression. These allosteric enzymatic nucleic acids or allozymes (see for example George et al, U.S. Pat. Nos. 5,834,186 and 5,741,679, Shih et al., U.S. Pat. No. 5,589,332, Nathan et al., U.S. Pat. No. 5,871,914) are designed to respond to a signaling agent, which in turn modulates the activity of the enzymatic nucleic acid molecule and modulates expression of JNK or AIF. In response to interaction with a predetermined signaling agent, the allosteric enzymatic nucleic acid molecule's activity is activated or inhibited such that the expression of a particular target is selectively down-regulated. The target can comprise JNK and/or AIF.

Target Sites. Targets for useful enzymatic nucleic acid molecules and antisense nucleic acids can be determined as disclosed in Draper et al., WO 93/23569; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Enzymatic nucleic acid molecules and antisense to such targets are designed as described in those applications and synthesized to be tested in vitro and in vivo, as also described. The sequences of human JNK or AIF RNAs can be screened for optimal enzymatic nucleic acid and antisense target sites using a computer-folding algorithm. While human sequences can be screened and enzymatic nucleic acid molecule and/or antisense thereafter designed, as discussed in Stinchcomb et al., WO 95/23225, mouse targeted enzymatic nucleic acid molecules can be useful to test efficacy of action of the enzymatic nucleic acid molecule and/or antisense prior to testing in humans.

The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Enzymatic nucleic acid molecule binding/cleavage sites are identified and designed to anneal to various sites in the RNA target. The binding arms are complementary to the target site sequences described above. The nucleic acid molecules are chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211, 3-19.

Synthesis of nucleic acids greater than 100 nucleotides in length can be difficult using automated methods, and the therapeutic cost of such molecules can be prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure.

Oligonucleotides (eg; antisense, GeneBlocs) are synthesized using protocols known in the art as described in Caruthers et al., 1992, Methods in Enzymology 211, 3-19; Thompson et al., International PCT Publication No. WO 99/54459; Wincott et al., 1995, Nucleic Acids Res. 23, 2677-2684; Wincott et al., 1997, Methods Mol. Bio., 74, 59; Brennan et al, 1998, Biotechnol Bioeng., 61, 33-45; and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer.

Inactive ribozymes or binding attenuated control (BAC) oligonucleotides can be synthesized. Similarly, one or more nucleotide substitutions can be introduced in other enzymatic nucleic acid molecules to inactivate the molecule and such molecules can serve as a negative control.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein). For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Perrault et al. Nature, 1990, 344, 565-568; Pieken et al. Science, 1991, 253, 314-317; Usman and Cedergren, Trends in Biochem. Sci., 1992, 17, 334-339; Sproat, U.S. Pat. No. 5,334,711; and Beigelman et al., 1995, J. Biol. Chem., 270, 25702; Beigelman et al., U.S. Pat. No. 5,716,824; Usman et al., U.S. Pat. No. 5,627,053; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity can not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of subjects with nucleic acid molecules can also include combinations of different types of nucleic acid molecules.

In one embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid can not be significantly lowered. As exemplified herein such enzymatic nucleic acids are useful in a cell and/or in vivo even if activity over all is reduced about 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure. By “cap structure” is meant chemical modifications, which have been incorporated at either terminus of the oligonucleotide (see for example Wincott et al, WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonyhnethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al, International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al, 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

Various modifications to nucleic acid (e.g., antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of subjects with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., 1992, Nucleic Acids Symp. Ser., 27, 15-16; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, lenti virus, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient or subject followed by reintroduction into the patient or subject, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operably linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al, 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

A further object of the present invention is to provide a kit comprising a suitable container, the therapeutic of the invention in a pharmaceutically acceptable form disposed therein, and instructions for its use.

It is understood that the examples and embodiments described herein are for illustrative purposes only and that various modifications or changes in light thereof will be suggested to persons skilled in the art and are included within the spirit and purview of this application and are considered within the scope of the appended claims. All publications, patents, and patent applications cited herein are hereby incorporated by reference in their entirety for all purposes.

The following detailed examples are given by way of example of the preferred embodiments, and are in no way considered to be limiting to the invention. For example, the relative quantities of the ingredients may be varied to achieve different desired effects, additional ingredients may be added, and/or similar ingredients may be substituted for one or more of the ingredients described. Additional advantageous features and functionalities associated with the systems, methods and processes of the present invention will be apparent from the examples which follow. EXAMPLE #1

### Activation Induced Cell Death of Human Melanoma Epitope Specific Primary Cytotoxic T Lymphocytes Mediated by the Release of Mitochondrial AIF

Materials and Methods

Study population, cell lines, culture medium and reagents. HLA-A2-positive melanoma patients or healthy donors were included in the study with informed consent. Culture medium, and other reagents used to generate MART-127-35 peptide specific CTL and to assess AICD have been described previously.

In Vitro CTL generation assay. Myeloid dendritic cells generation from peripheral blood, and in vitro CTL generation assay were carried out as described previously. Briefly, CD8+ T cells, purified from PBMC were co-cultured with autologous DCs, pulsed with relevant peptides (100 μg/ml) and 5 μg/ml of β2-microglobulin, in the presence of rhIL-15 (100 ng/ml).

AICD induction and quantitation. AICD in the human primary CTL primed and expanded specifically for melanoma epitope MART-1₂₇₋₃₅ was induced by re-exposure of the CTLs to MART-1₂₇₋₃₅ peptide (1 μg/ml) as reported previously. Staurosporine mediated death was induced by exposure of CTL to 1 μM staurosporine for 4 hr. Quantitation of AICD and the effects of various inhibitors on AICD of CTLs, were carried out by annexin-V staining followed by FACS analyses as reported previously.

DNA fragmentation analysis. DNA fragmentation was assessed by measuring single stranded nick generation, using single stranded DNA nick specific antibody (Alexis Biochemicals, CA, USA) with flow-cytometry and immunofluorescence according to published protocols. Antibody-stained cells were counterstained with 0.1 μg/ml DAPI in PBS for 10 min. Agarose gel electrophoresis was used to detect small oligosomal DNA fragmentation and pulse field electrophoresis (Bio-Rad CHEF-DRIII) was used (6 V/cm, 20-60 sec pulse overnight) to detect large-scale DNA fragmentation.

Sub-cellular Fractionation. Sub-cellular fractionation was done according to Gross, et al., with minor modifications. Heavy membrane and light membrane fractions were solublized in RIPA buffer supplemented with protease inhibitors and the proteins extracted from heavy membrane (HM), light membrane (LM) and supernatant (S-100) fractions were analyzed by immuno-blot analysis.

Immunoblot analysis. Proteins were extracted with RIPA buffer, resolved on 15% SDS polyacrylamide gels, and electrophoretically transferred to PVDF membranes (Millipore, USA). Blots were probed with appropriate antibodies and developed using a chemiluminescence reagent (Amersham Biosciences, NJ, USA) as previously reported. For immunoprecipitation experiments, 50-100 micrograms of protein samples were incubated with 4-5 micrograms of primary antibodies and protein A- or G-Sepharose resin (Amersham Biosciences, NJ, USA) at 4° C. for 45 min. Proteins bound to the resin were washed three times with RIPA buffer, re-suspended in sample buffer, and analyzed by SDS-PAGE and immunoblotting.

Immunofluorescence microscopy. The MART-1₂₇₋₃₅ specific primary CTL (2×10⁵ cells) were deposited onto glass cover slips by cytospin. The cells were fixed with 4% para-formaldehyde in PBS and stained with appropriate antibodies for co-localization of proteins by fluorescence microscopy. Fluorescent microscopic images were acquired using the Olympus ×70 microscope equipped with a 100× 1.3 NA UPlanFI objective and a Hamamatsu Orca-ER CCD camera. Image acquisition and analysis were carried out using the OpenLab 3.1.1 software and Adobe Photoshop 7.0.

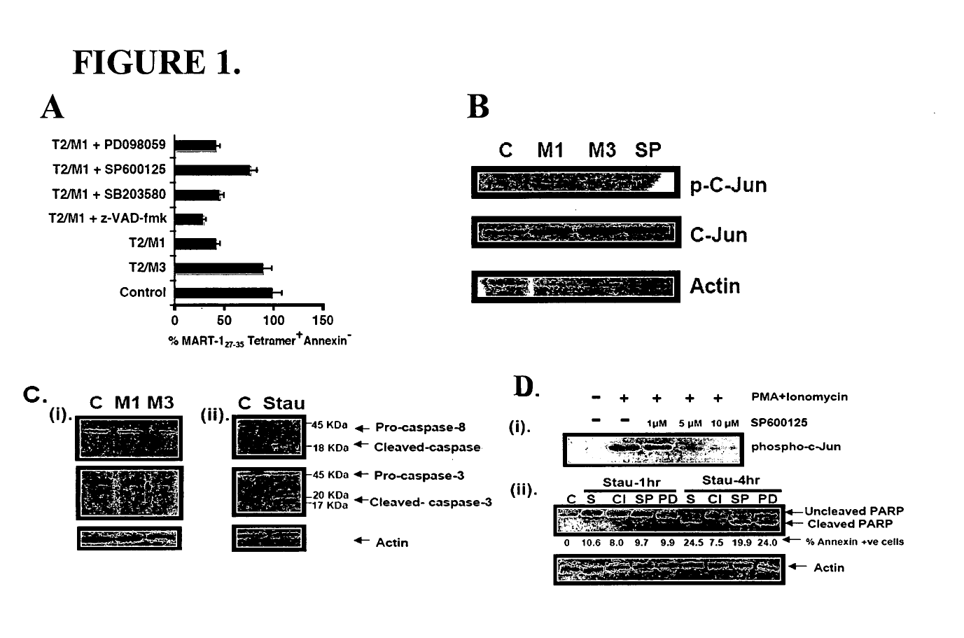

FIG. 1 demonstrates the the protective affect of the JNK inhibitor SP600125 on AICD in MART-1₂₇₋₃₅ epitope specific CTL. While the caspase inhibitor (z-VAD-fmk), p38 inhibitor (SB203580) and ERK1/2 MAP kinase inhibitor (PD98059) could not protect the primary CTL from AICD, the JNK inhibitor, SP600125, protected the primary CTL from AICD (FIG. 1A). FIG. 1B shows that c-Jun was activated in these CTL during AICD and SP600125 blocked c-Jun activation. Neither caspase-8 nor caspase-3 were activated in these CTL encountering the cognate epitope (FIG. 1C). Staurosporine was used to induced death (prototypic caspase-induced cell death) to verify the effectiveness of the pan-caspase inhibitor, z-VAD-fmk, as a control. FIG. 1D shows the inhibitory effect of SP600125 on c-Jun activation and also shows that the PARP cleavage induced by saturosporine was blocked by the pancaspase inhibitor, z-VAD-fmk. FIG. 1D also shows that the pancaspase inhibitor z-VAD-fmk protected cells from staurosporine-induced death.

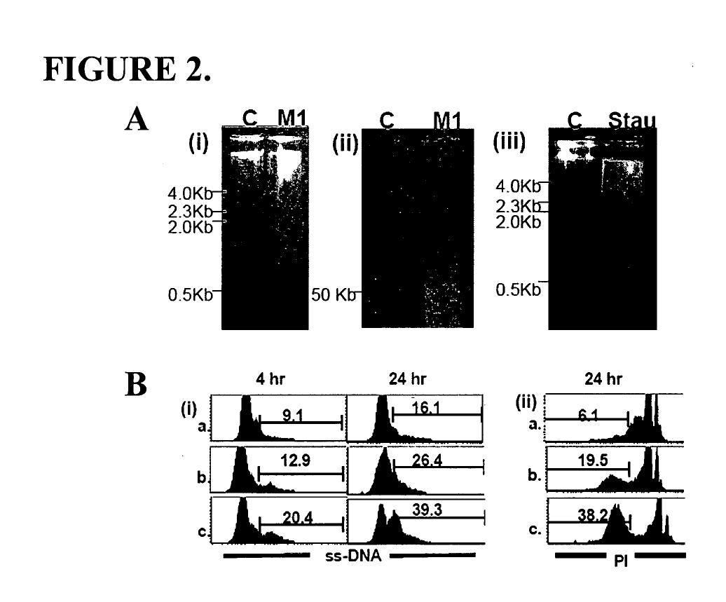

Caspase-dependent apoptosis of T cells is associated with oligosomal DNA fragmentation. Since AICD in these CTL was not blocked by the pan-caspase inhibitor (FIG. 1A), we examined the nature of the chromatin degradation in these CTL during AICD. As shown in FIG. 2A, standard agarose gel electrophoresis revealed no oligosomal DNA fragmentation in the CTL exposed to the MART-1₂₇₋₃₅ epitope (M1). In contrast, pulse-field-gel-electrophoresis revealed large-scale DNA fragmentation (˜50 Kbp) in these CTL encountering the same epitope (FIG. 2A). Oligosomal DNA fragmentation was, however, clearly evident in staurosporine-induced death in these CTL (FIG. 2A iii). We further characterized the nature of DNA degradation by looking for ssDNA nicks in the CTL undergoing AICD with an antibody that recognizes nicked DNA. CTL undergoing AICD stained strongly for nicked ssDNA. Flow cytometric analyses provided a quantitative confirmation of the microscopic observation and also showed a corresponding increase in PI stained sub-diploid dead cells (FIG. 2B). These observations revealed that chromatin damage in AICD in primary CTL is large-scale DNA fragmentation (characterstic of caspase-independent mitochondrial cell death) as opposed to oligosomal DNA breaks (characteristic of caspase-dependent apoptosis).

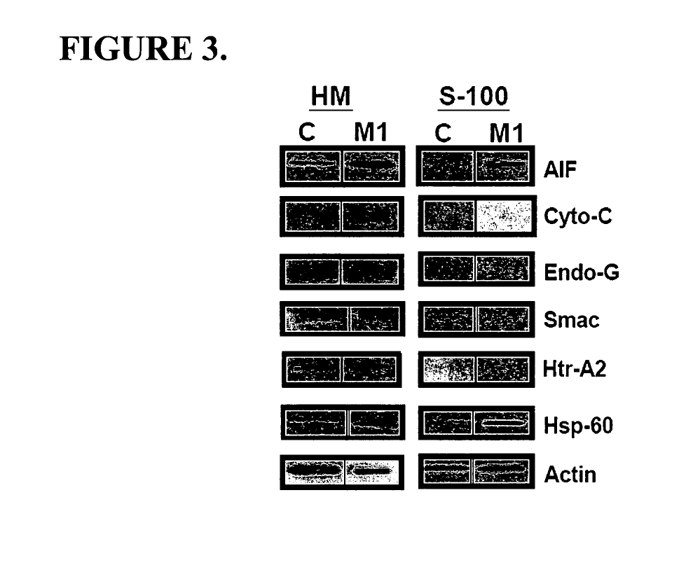

AIF, a mitochondria-resident apoptogenic protein, causes ssDNA breaks in a caspase-independent manner after being released from mitochondria. In contrast, in caspase-dependent apoptosis, cytochrome-c, another mitochondrial apoptogenic protein, is released with the eventual activation of caspase-3 via the activation of caspase-9. Accordingly, we asssayed if either of these two factors (cytochrome-c or AIF) is released from mitochondria in the primary CTL during AICD. Heavy membrane (HM) fractions were isolated and S100 fractions from CTL undergoing AICD and steady state control CTL, and probed for the various pro-apoptotic factors. As shown in FIG. 3, no cytochrome-c release was detected in the cytosolic fraction of CTL during AICD, but AIF was released into the cytosolic fractions of CTL undergoing AICD (FIG. 3). Of interest, other mitochondrial pro-apoptogenic factors such as Endo-G, Smac, and HtrA-2 were either minimally or not at all released into the cytosolic fractions during AICD.

Since AIF was selectively released into the cytosolic fraction of the CTL undergoing AICD and large-scale (50kbp) DNA fragmentation was associated with mitochondrio-nuclear translocation of AIF, evidence of such translocation in the CTL during AICD was assayed. Immunohistochemical analyses revealed AIF to be primarily localized outside the nuclei in the control CTL. However, AIF clearly translocated to the nuclei in the CTL undergoing AICD. The mitochondrio-nuclear translocation of AIF was blocked by the JNK inhibitor SP600125, consistent with its protective effect on AICD of these CTL (FIG. 1A). The ERK1/2 inhibitor, PD98059, could neither block AIF translocation nor could prevent these CTL from undergoing AICD (FIG. 1A). Immunohistochemical analyses of CTL undergoing AICD over time revealed breakdown of nuclear chromatin into nuclear bodies and AIF was found to co-localize with these nuclear bodies. AIF was localized at the single stranded nick sites in CTL undergoing AICD at single cell level. These results confirmed that the AICD in these primary CTL was induced by ssDNA nicks resulting from the release of AIF without any release of cytochrome-c, and supported a critical role for JNK in this process.

The prevailing paradigm for AICD has been that it is triggered by the engagement of extrinsic death receptors (DR) such as Fas or TNFR and that caspases execute the cell death. Caspase-independent apoptosis, in general, and a DR-independent and caspase-independent mechanism of AICD of CTL, in particular, has been described, but the molecular mechanism behind this process remains unclear. In this context, it should be pointed out that the early seminal studies that described oligosomal DNA fragmentation in AICD were carried out either with immature T cells or with mouse T cells following TCR ligation and IL-2, following Fas ligation in activated T cells, or following TNF treatment. However, we could not fmd a careful examination of the nature of the DNA fragmentation during AICD solely from TCR ligation in antigen specific human primary CTL.

In these studies, a critical examination of the mechanism underlying AICD in a human tumor associated antigenic epiotpe specific primary CTL was explored. First, the data confirm that AICD in primary CTL is not associated with the activation of caspase-3 and caspase-8 and reveal that AICD in these CTL does not result from oligosomal DNA fragmentation—one of the hallmarks of caspase-mediated apoptosis (FIGS. 1 & 2). Instead, AICD in these CTL is associated with large scale DNA fragmentation (FIG. 2). Second, the absence of cytochrome-c release and the release of AIF from mitochondria suggest a novel mechanistic basis of the cell death. Third, the blocking effect of SP600125 on the mitochondrio-nuclear translocation implicates JNK in the regulation of the apoptotic process in these CTL. The precise mechanism underlying the selective AIF release (without any cytochrome-c release) remains to be clarified. Nonetheless, the study provides new insights into AICD, reveals the existence of a novel mitochondria-based death pathway in the AICD in primary CTL, and identifies new targets for interfering with the death pathway in ACID. As such, the findings have implications in active specific or adoptive immunotherapy for cancer.

### EXAMPLE #2

### Effect of siRNA Knockdown of JNK and AIF in AICD in Jurkat Cells

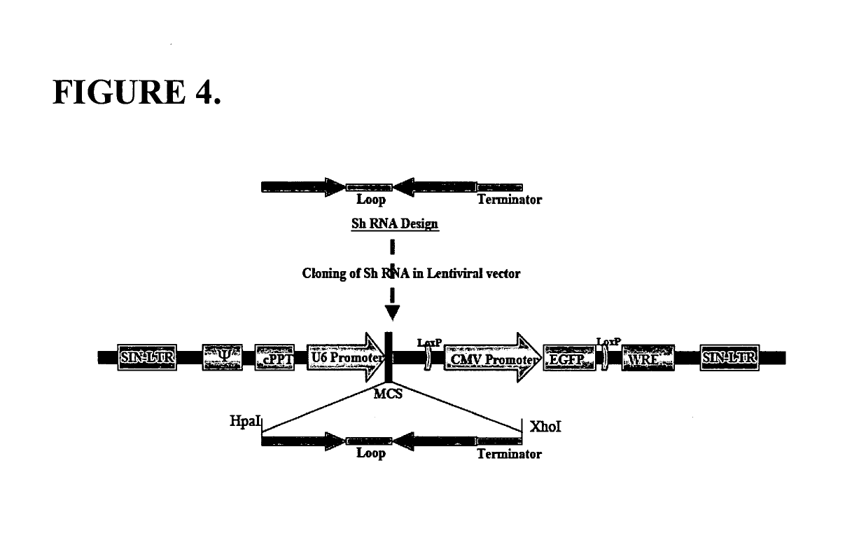

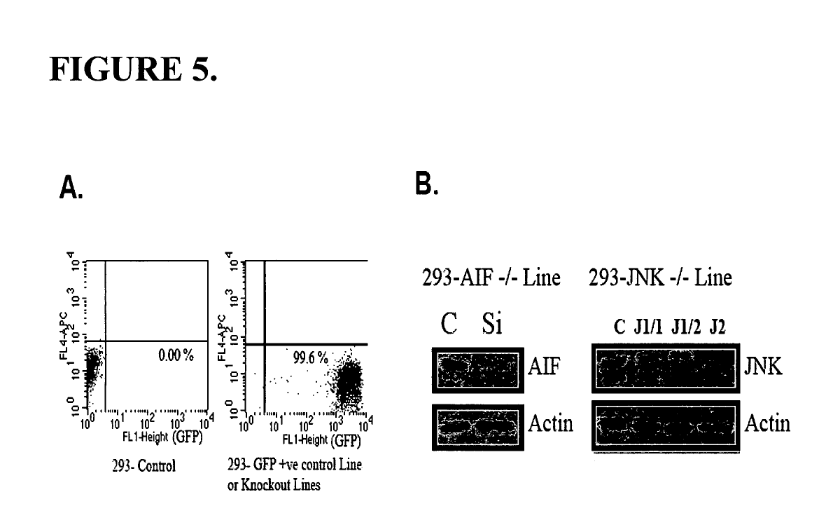

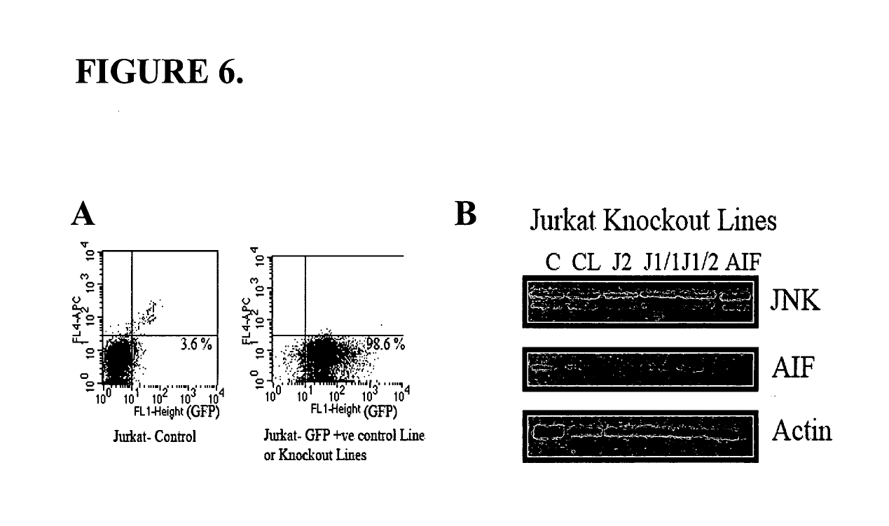

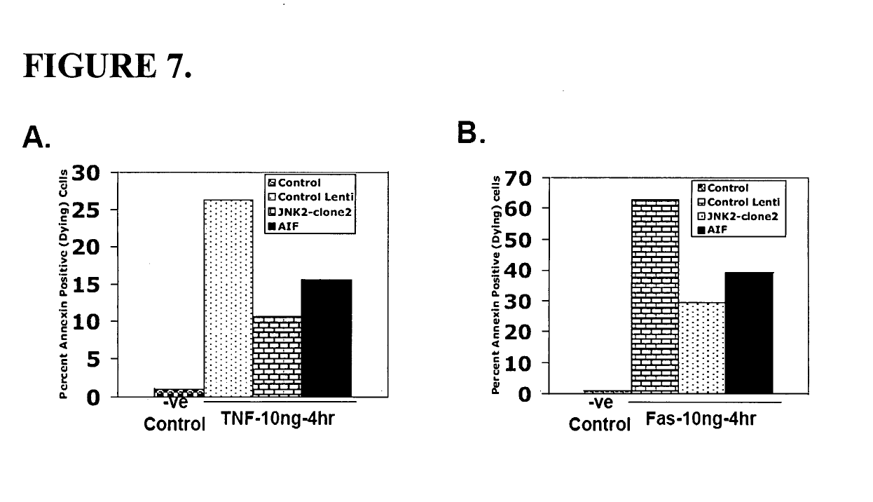

Data indicates a regulatory role of JNK in AIF-mediated AICD in primary CTL as well as in Jurkat cells. Therefore, interfering RNA-induced silencing of the respective genes encoding JNK and AIF was assessed. For the purpose, JNK and AIF knockdown cell lines were created comprising an siRNA expression vector encoding an siRNA, for example, a short hairpin RNA (shRNA). Selected sequences were designed in short hairpin format, shown schematically in FIG. 4 and cloned in a lenti-viral plasmid vector. The shRNA sequences were designed to target JNK and AIF genes. In a preferred embodiment the siRNA sequences correspond to SEQ ID NO:1, and 2 for JNK and SEQ ID NO:3 for AIF. Packaging of viruses was done in HEK-293 cells by co-transfecting the recombinant lenti-viral vector, helper plasmid and plasmid encoding VSV-G protein. Packaged viruses were used for infecting HEK-293 cells. Since the lenti-viral vector expresses the GFP gene, infection efficiency could be easily tracked. Infected cells were serially diluted and GFP positive clones were picked up. GFP positive clones were further propagated into 100% GFP positive cell lines, which were then tested for knockdown of desired genes (JNK and AIF). FIG. 5 and 6, shows efficient knockdown of JNK and AIF genes in lines generated. A similar strategy was employed to successfully create JNK and AIF knockdown lines of Jurkat cells, human leukemic T-cell line (E6; American Type Culture Collection, Manassas, Va.) (FIG. 5B). Preliminary experiments have shown that the JNK and AIF silenced Jurkat cells are protected against death from the ligation of the death receptor (Fas and TNFR). Similar methods can be employed in primary CTLs.

### EXAMPLE #3

### Rescuing Melanoma Epitope Specific Cytolytic T Lymphocytes from Activation Induced Cell Death, by SP600125, an Inhibitor of Jun N-Terminal Kinase.

Materials and Methods

Study Population

The study population consisted of HLA-A 2 positive melanoma patients or healthy donors. The participants were included in this study with informed consent.

Culture Medium and Reagents

The MART-1₂₇₋₃₅ peptide (AAGIGILTV) and MAGE-3₂₇₁₋₂₇₉ (FLWGPRALV) was purchased from Multiple Peptide Systems, San Diego, Calif. while β2-microglobulin was purchased from Sigma (St. Louis, Mo.). Culture medium consisted of Iscoves Modified Dulbecco's Medium (GIBCO BRL, Grand Island, N.Y.) supplemented with 10% fetal bovine serum (FBS) (Gemini Bioproducts Inc., Calabasas, Calif.), 0.55 mM L-arginine, 0.24 mM L-asparagine (both from GIBCO BRL, Grand Island, N.Y.), 1.5 mM L-glutamine (Sigma, St. Louis, Mo.), 50 U/ml penicillin and 50 μg/ml streptomycin (both from Abbott Laboratories, North Chicago, Ill.). This will be referred to as complete media (CM). The TAP-deficient line, T2, was a gift of Peter Cresswell (Yale University, New Haven, Conn.). Recombinant human granulocytes macrophage colony-stimulating factor (GM-CSF) was purchased from Immunex, Seattle, Wash. Recombinant human interleukin rhIL-4, rhIL-2, rhIFN-γ was purchased from R & D Systems, Inc., Minneapolis, Minn.). Lipopolysaccharide from E. coli 055: B5 was purchased from Sigma (St. Louis, Mo.). Annexin V kit to track the early apoptotic cells for exposure of phosphatidylserine was purchased from BD Pharmingen, San Jose, Calif. MART-1₂₇₋₃₅ (EAGIGILTV) tetramer labeled with phycoerythrin (PE) with and without fluorescein isothiocyanate (FITC) labeled anti CD8 was purchased from Beckman Coulter Inc., Fullerton, Calif. Flurochrome-labeled monoclonal antibodies (mAbs) to CD 25, CD 27, CD 28, CD 95, CD 95L, 4-1BB, 4-1BBL, OX-40 were purchased from BD Biosciences, San Jose, Calif. Inhibitors for various kinase pathways, as SB203580 for p38 kinase, SP600125 (anthra[1,9-cd]pyrazol-6(2H)-one) for JNK, PD98059 for Erk were purchased from Biomol International, PA. Pan caspase inhibitor, human Fas/Fc chimera, human TNF-RI/Fc chimera, human TRAIL-RI/Fc chimera, human TRAIL-RII/Fc chimera and human IFN-γ RI/Fc chimeric proteins were purchased from R & D Systems, MN.

Generation of Dendritic Cells from Peripheral Blood Monocyte

The procedure for generating myeloid dendritic cells from peripheral blood monocyte was followed. Briefly, circulating monocyte were isolated by 2 h adherence of Ficoll-Hypaque density gradient-cut PBMC as previously described. The adherent cells were cultured in CM with 1000 U/ml GM-CSF and 500 U/ml IL-4 for 3-5 days to obtain a population of immature DCs (iDCs). Maturation of iDCs was done by first priming in IFN-γ (1000 U/ml) for 2 h and then in presence of 100 ng/ml lipopolysaccharide (LPS).

IFN-γ Response Assay

IFN-γ response assay for the effector cells has been described previously. Briefly, the effector cells were co-cultured with the peptide pulsed (1 μg/ml) T2 cells. After 4-16 h, culture supernatants were harvested and IFN-γ was measured by ELISA as per manufacturer's protocol (R & D Systems, MN).

FACS Analysis

Procedure for phenotypic analyses and for determining the number of epitope specific cells with tetramer staining by flow cytometry has been described. To determine tetramer and annexin V positive cells, the effector cells were stained with CD8, MART-1₂₇₋₃₅/HLA-A2 tetramer, and Annexin V. The stained cells were then analyzed for single positive vs. double positive populations in flow cytometry using a FACSCalibur and the Cellquest software (Becton Dickinson, San Jose, Calif.).

Microcytoxicty Assay

The chromium release microcytotoxicity assay has been previously described.

Activation of CD 8⁺ T cells by DC-Based Presentation of Epitopes, in Vitro

The basic procedure for peptide-loaded DC-based in vitro activation and expansion of epitope specific CD 8⁺ T cells has been described. Briefly, Ficoll-Hypaque gradient separated blood mononuclear cells were purified for CD 8⁺ T cells (routinely exceeding 90%) by Dynal magnetic bead isolation kit (Dynal, Oslo, Norway) and co-cultured with autologous DCs pulsed with relevant peptides [100 μg/ml] and 5 μg/ml of β2 microglobulin at a CD 8⁺ T cell to DC ratio of hundred. For our current purpose, since IL-2 has been known to facilitate apoptosis in activated T cells, the co-cultures were carried out in the presence of rhIL-15 (10 ng/ml). Prior to setting up co-cultures, the DCs were irradiated to 3000 rad. The activated CTLs were also maintained in culture in IL-15.

Assay for AICD Induction

In test whether or not the epitope specific primary CTLs undergo AICD, the activated CTLs were exposed to peptide [1 μg/ml] loaded T2 cells (Effector: Target=100) at different time points of the cultures. Thereafter, the evidence of apoptosis was determined by flow cytometry with triple color staining (CD8, MART-1₂₇₋₃₅/HLA-A2 tetramer and annexin V) at different time points (4-18 h). Experiments were carried out in triplicate wells and significance was calculated by One-way analysis of variance (ANOVA) using Sigma Stat statistical software, Chicago, Ill.

To evaluate the effect of various agents in modulating AICD, the CTLs were pre-incubated with various compounds at optimal concentration for 45 min. at 37° C. and then exposed to T2 cells alone or loaded with peptide. The optimal dose used in the experiments shown in this paper was determined by using these compounds at different concentrations in the preliminary experiments (data not shown).

Western Blot Analysis

Washed cell pellets were lysed in RIPA buffer and a cytosolic fraction was prepared by spinning the lysate at 15,000 rpm. Proteins (20 μg/well) were separated on 15% SDS-PAGE, transferred onto nitrocellulose filters, probed with appropriate antibodies. Antibodies for Bcl-2, phospho Bcl-2, Bim, BCI-X_{L}, Bax, Actin were purchased from Santa-Cruz Biotechnology, Santa-Cruz, Calif. Anti-Mcl-1 antibody was purchased from BD Pharmingen, Palo Alto, Calif. Blots were developed by HRP-tagged secondary antibody (Santa-Cruz Biotechnology, Santa-Cruz, Calif.) using a chemoluminescence kit (Amersham Biosciences, Buckinghamshire, U. K.).

JNK Activation Assay

JNK activation assay was performed using a JNK activation assay kit (Calbiochem, San Diego, Calif.) as per the manufacturer's protocol.

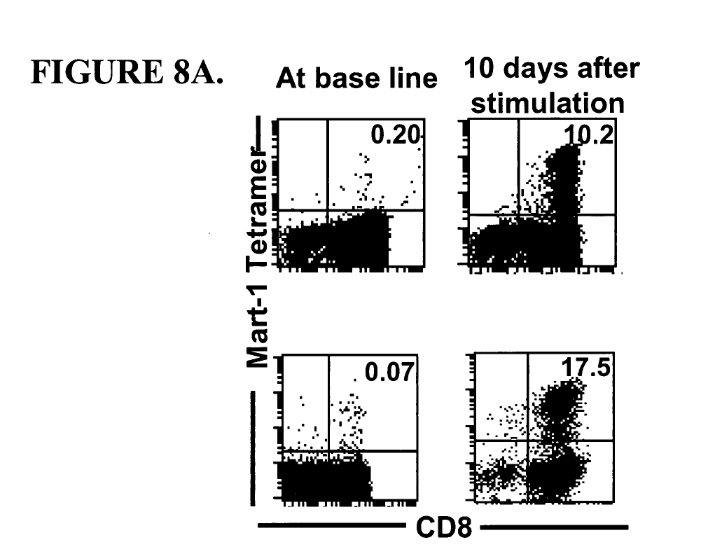

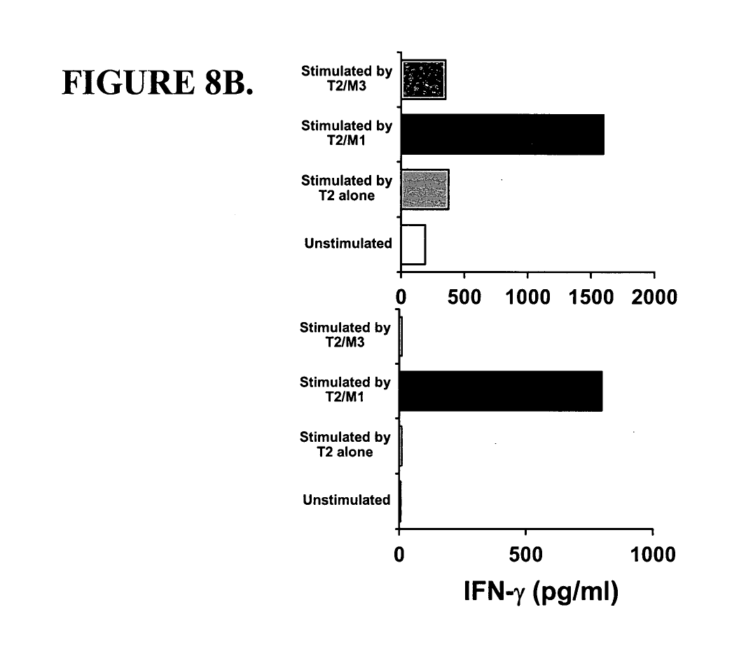

Donors were selected (healthy donors as well as melanoma patients) who harbor MART-1₂₇₋₃₅ epitope specific CTLs in relatively high frequencies and whose CTL precursors (CTLp) could be easily activated. Because IL-2 has been implicated in apoptosis in activated T cells, we looked for an alternative cytokine support in the in vitro CTL generation protocol. We found that recombinant IL-15 supports the activation and expansion of the epitope specific CTLs in the mDC-based epitope presentation system. IL-15 not only supports the primary activation process, but also supports survival of the activated T cells in an antigen independent manner. IL-15Rα is not down regulated after T cell activation, and IL-15 has an important role in T cell memory generation as such, as well as in T cell survival. All experiments were carried out in the presence of IL-15. The MART-1₂₇₋₃₅ epitope specific CTLs were readily activated and expanded when they were stimulated by peptide pulsed matured DCs in the presence of IL-15, in vitro. FIG. 8A shows an example of the expansion of the MART-1₂₇₋₃₅ epitope specific CTLs derived from a normal healthy individual and a melanoma patient. The expanded MART-1₂₇₋₃₅ epitope specific cells also exhibited IFN-γ response in an epitope specific manner (FIG. 8B).

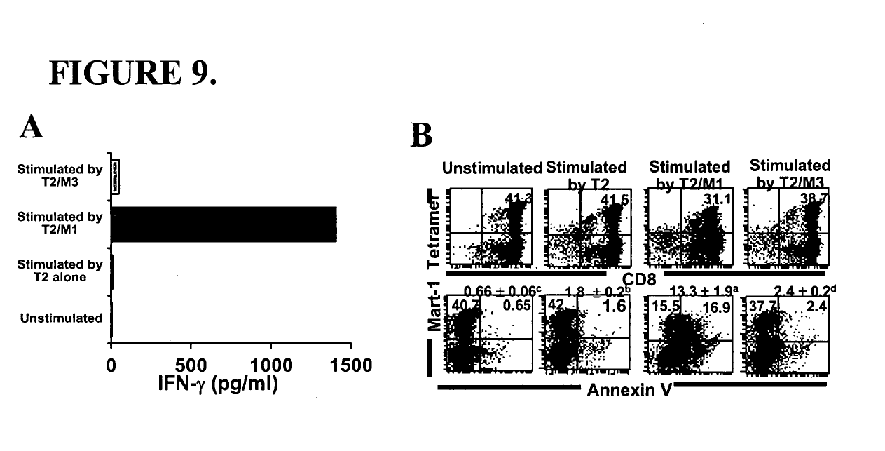

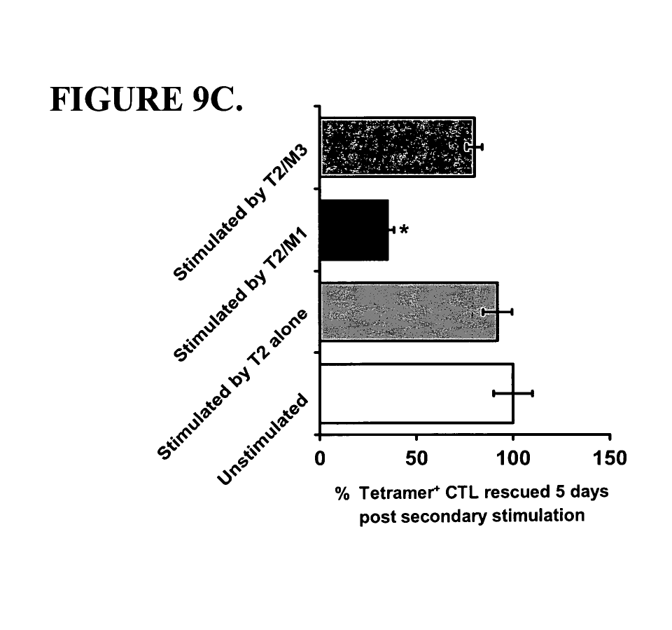

The fate of the MART-1₂₇₋₃₅ epitope specific CTLs upon secondary encounter of the cognate epitope, (i.e., whether following effector function, they survive or die) was then examined. The CTLs and the peptide pulsed T2 cells were co-cultured in CM without any cytokine and 4 h later the evidence of early death was examined by annexin V staining. As shown in FIG. 9, although the CTLs were fully functional in IFN-γ assay (FIG. 9A), a large fraction of these activated CTLs became annexin V positive at 4 h (FIG. 9B). When the co-cultures were continued in IL-15 for five days and the number of epitope specific CTLs were counted, only 20-30 % of the starting population could be recovered from the co-culture of the CTLs with the cognate target. An example for loss of the epitope specific CTLs following effector function is shown in FIG. 9C. Thus, about 50% of the antigen specific population showed early evidence of death at 4 h (FIG. 9B) and a much larger fraction eventually died following secondary encounter of the cognate epitope.

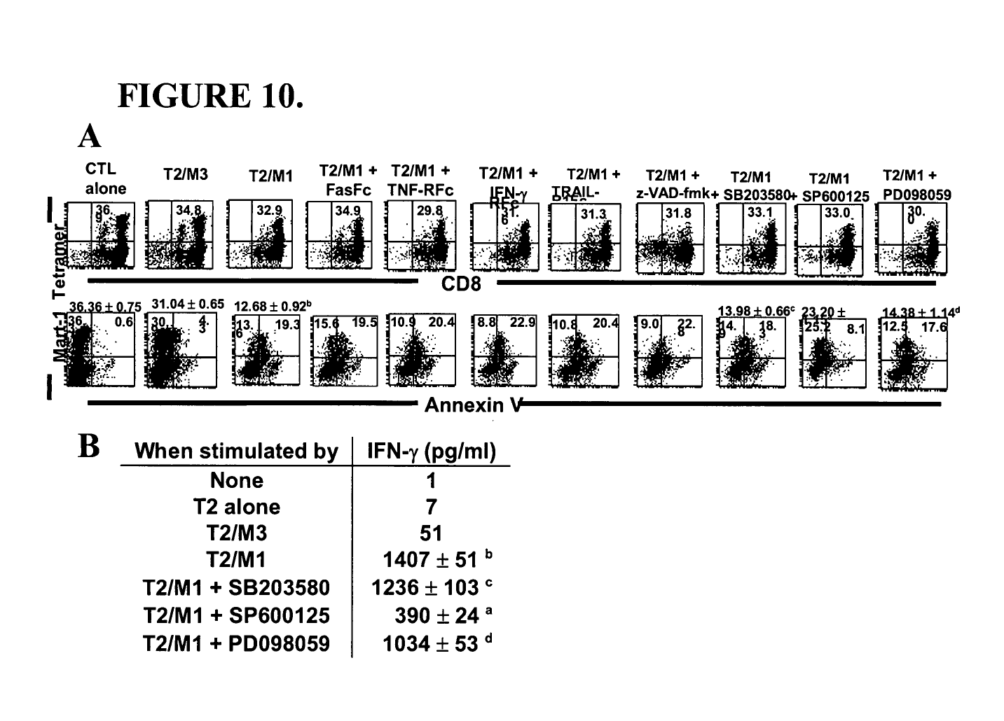

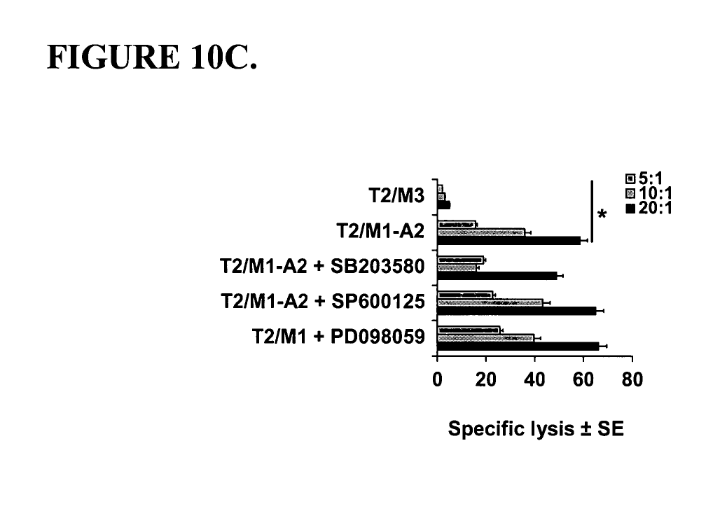

The mechanism of AICD in these CTLs was examined to determine if these CTLs could be rescued from AICD by interfering with the death signaling pathway(s). We examined the effect of interfering with the external death signal receptors (such as FAS, TNF-R, TRAIL, etc.) on AICD, and whether any of the MAP kinase inhibitors could block the AICD in these CTLs since MAP kinases have been implicated in the negative selection of developing thymocytes. As shown in FIG. 10A, the death of these CTLs was not caspase dependent, as the pan caspase inhibitor, z-VAD-fink did not prevent apoptosis. The apoptosis was not also affected by the blockade of the common extrinsic death signal receptors such as FAS, TNF-R or TRAIL. However, while the p38 and ERK inhibitors had no effect, the JNK inhibitor, SP600125, rescued a significant fraction of the CTLs from AICD. Interestingly, SP600125 also inhibited the IFN-γ response by these CTLs (FIG. 10B) but did not affect their cytotoxic function (FIG. 10C) suggesting that the cytolytic machinery and the IFN-γ response pathways are differently regulated in these CTLs. Induction of AICD upon secondary exposure to the cognate epitope in the primary CTLs and the protective effect of SP600125 from AICD were observed in CTLs generated from two normal donors and two melanoma patients (collective data not shown).

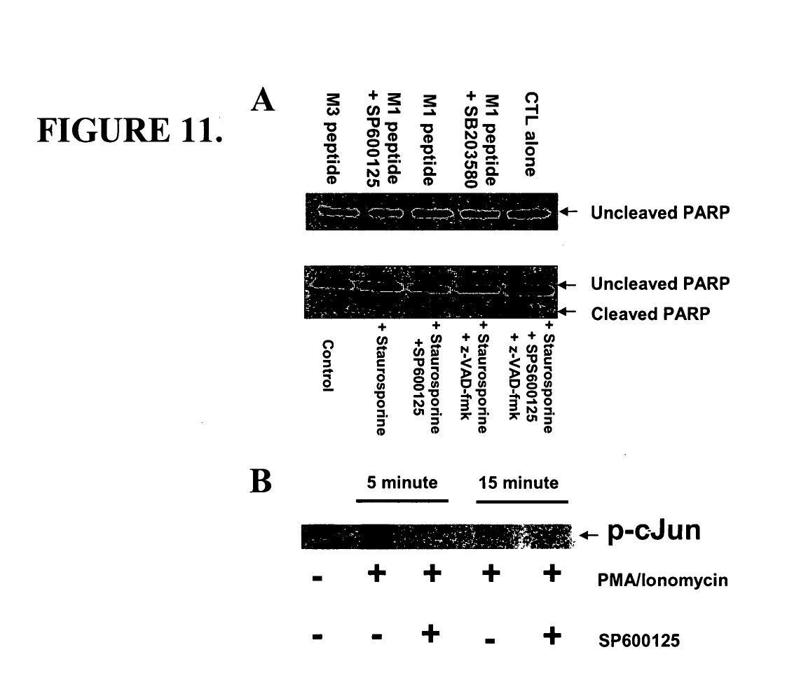

The caspase-independent death in these primary CTLs upon TCR engagement was confirmed as PARP was found to be uncleaved (FIG. 11A). Of note, PARP was cleaved in staurosporine treated Jurkat cells and z-VAD-fink blocked the PARP cleavage. SP600125 also abrogates c-jun activation (FIG. 11B).

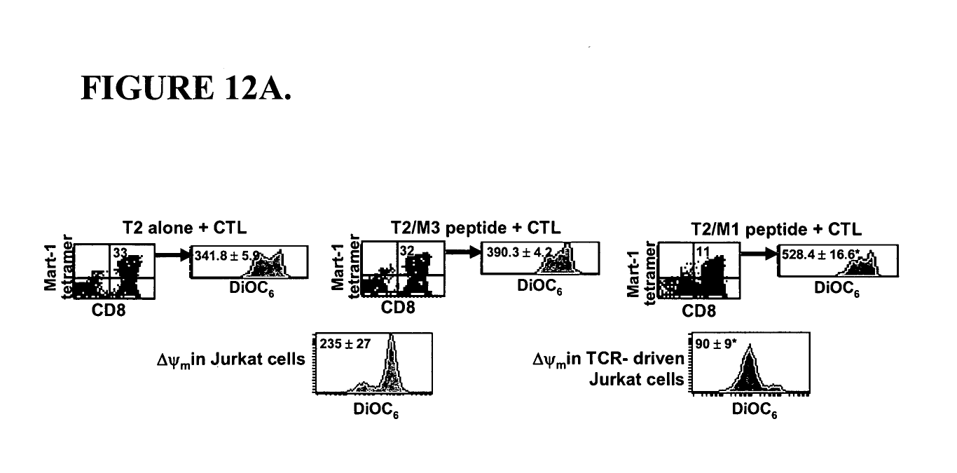

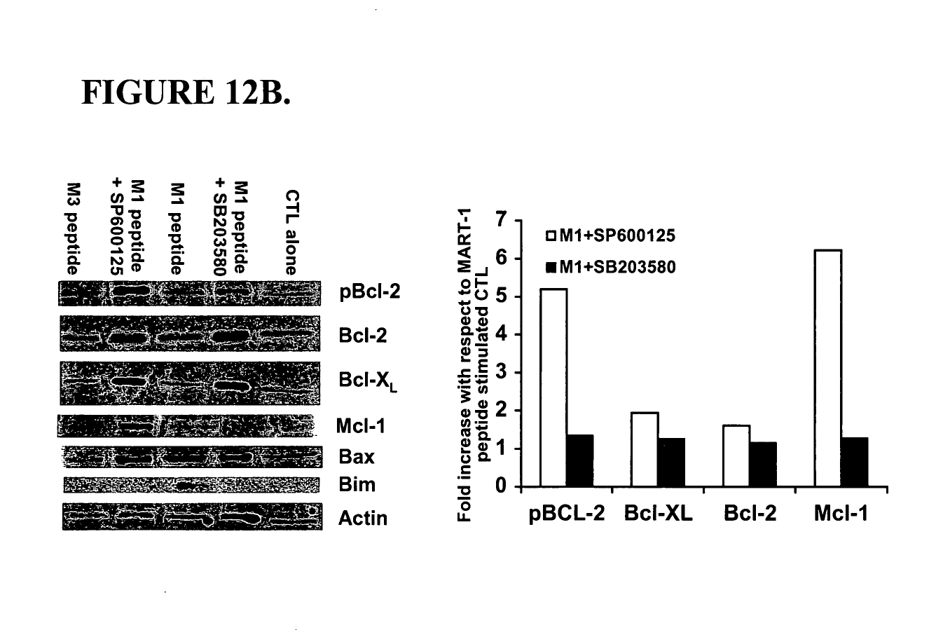

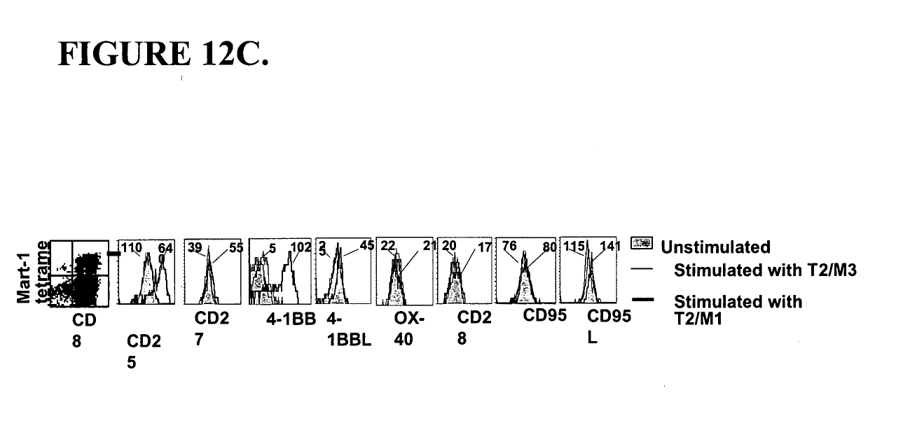

The status of the mitochondrial membrane potential was examined in the CTLs during AICD. As shown in FIG. 12A, the CTLs surprisingly exhibited hyper polarization of the mitochondrial transmembrane potential (Δψₘ) while the apoptotic Jurkat cells exhibited hypo polarization of the mitochondrial membrane. Hyper polarization has been associated with apoptotic process in peripheral blood lymphocytes and has also been shown to occur independently from activation of caspases. Further, hyper polarization of mitochondrial membrane potential has been shown to be a reversible stage in mitochondrial death decision process while hypo polarization usually represents a point of irreversible commitment to death. Interestingly, SP600125 (JNK inhibitor) as well as SB203580 (p38 inhibitor) decreased the level of hyper polarization induced by the re-stimulation with the cognate peptide (data not shown) suggesting that SP600125 mediated rescue is not specifically mediated through the modulation of mitochondrial membrane potential.

The status of Bcl family pro-and anti-apoptotic proteins and co-stimulation through certain T cell receptors (CD28, 4-1BB, OX-40, etc.) influence survival of activated T cells. As such, the expression levels of the anti-and pro-apoptotic Bcl family proteins was examined, and these receptors in the CTLs in condition that induces AICD. As shown in FIG. 12B, while Bcl-2 and BCI-X_{L} were non-selectively up regulated by SB203580 and SP600125, only phosphorylated Bcl-2 and Mcl-1 were selectively up regulated by the JNK inhibitor SP600125. Bim, a pro-apoptotic protein, was markedly up regulated (or released from sequestered sites) by the CTLs undergoing AICD. Of interest, the CTLs up regulated 4-1BB (ten to twenty fold increase of the geometric mean intensity of fluorescence) and CD25 (three to six fold increase of the geometric mean intensity of fluorescence) upon secondary encounter of the antigen (FIG. 12C).

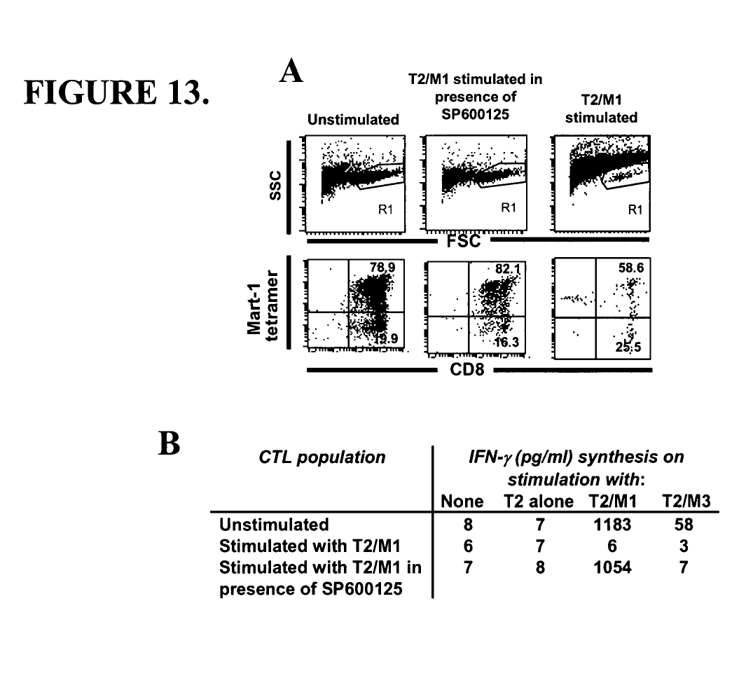

Finally, the functional status of SP600125 rescued CTLs upon continuous culture was examined. After overnight exposure of the CTLs to the cognate epitope in the presence or absence of SP600125, the CTLs were washed and then maintained in continuous culture in IL-15 containing CM without the inhibitor. Five days after, the number of viable MART-1₂₇₋₃₅ tetramer positive cells were determined and their function was tested in the IFN-γ response assay. FIG. 13A shows that only a small fraction of the starting population could be recovered from the cognate epitope exposed culture in the absence of the JNK inhibitor. A larger fraction of the starting epitope specific population was recovered from the co-culture that was started in the presence of the JNK inhibitor, SP600125. Remarkably, the rescued CTLs regained their capacity to synthesize IFN-γ (FIG. 13B).

The results are noteworthy for a number of reasons. First, the data show that a large fraction of the epitope specific primary CTLs generated, in vitro/ex vivo, are indeed susceptible to AICD from their very first re-encounter of the antigen even during the amplification phase (FIG. 9). It should be mentioned that the MART-1₂₇₋₃₅ epitope specific populations expanded more than a thousand fold in IL-15 without a second round of stimulation and then senesced around day fifty. These CTLs, interestingly, exhibited propensity to undergo AICD as early as on day 9 (collective data not shown). This, therefore, suggests that the apoptotic program in this in vitro CTL activation system is turned on quite early and lends support to the recent observations that the apoptotic program in viral antigen specific CTLs gets activated substantially before the contraction phase, in vivo. Of interest, stimulation of the CTLs with the cognate peptide that induced apoptosis, paradoxically up regulated the expressions of IL-Rα (CD25) and 4-1BB (CD 137) substantially (FIG. 12C). While these molecules are associated with T cell growth and survival, 4-1BB has lately been implicated in apoptosis and has also been shown to interfere with IFN-γ response but not with cytotoxicity in CTLs. A connection between 4-1BB-mediated signaling and activation of the JNK pathway has also been suggested. Second, the MART-1₂₇₋₃₅ epitope specific CTLs generated in the in vitro DC-based epitope presentation protocol in the presence of IL-15 and maintained in IL-15 were just as susceptible to AICD as the CTLs that were generated and maintained in IL-2 (collective data not shown).

Third, the results clearly reveal that SP600125 is capable of protecting these CTLs from AICD (FIG. 10). Since SP600125 functions as an inhibitor of the c-jun N-terminal kinase, JNK, the data suggest a role for JNK in the apoptotic process. Fourth, while SP600125 negatively regulates the IFN-γ responsiveness of the CTLs, it does not affect their cytotoxic function, suggesting that the two functional pathways in CTLs are differently regulated. Finally, and most importantly, the rescued CTLs regained IFN-γ responsiveness in continued culture after the inhibitor being washed off.

As mentioned earlier, Bcl family pro- and anti- apoptotic proteins play an important role in the survival or death of cells. Our observation on the modulation of some of these proteins, therefore, is of interest. The JNK inhibitor, SP600125, increased the level of pBcl-2 and Mcl-1. The effect of phosphorylated Bcl-2 on apoptosis of T cells remains an unsettled issue. However, Mcl-1 is an important Bcl family member with positive effect on T cell survival. It is therefore tempting to suggest that JNK inhibitor SP600125 prevents AICD in these CTLs by up-regulating Mcl-1.

### EXAMPLE #4

### Administration of SP600125 Blocks the Release of Apoptosis Inducing Factor (AIF) in Activated Cytolytic T Lymphocytes

We have found that a large fraction of melanoma epitope specific primary CTLs, generated in vitro, undergo activation induced cell death (AICD) after encountering their cognate epitopes. We have also found that the AICD in these CTLs is not triggered by the usual external death receptor (DR)-mediated signaling and is not caspase-dependent. Significantly, our studies indicated that these CTLs could be rescued from AICD by the c-jun N terminal kinase (JNK) inhibitor, SP600125. Given that the effectiveness of the CTLs can be considerably improved by protecting them from this type of death and based on our recent findings, we examined the mechanism underlying AICD in the melanoma epitope specific CTLs.

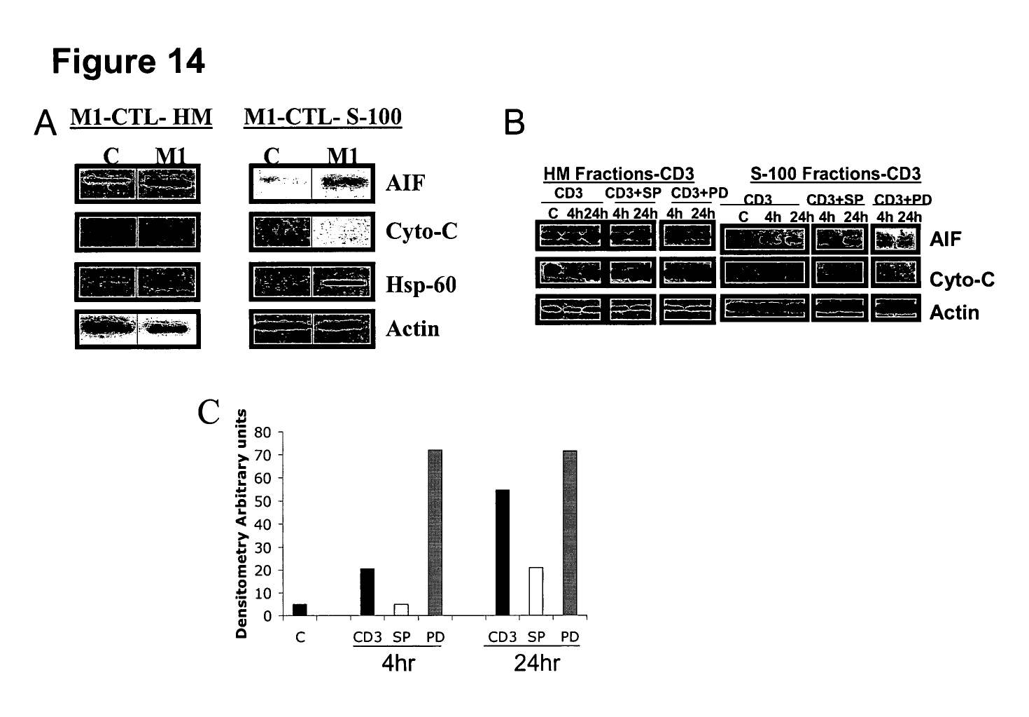

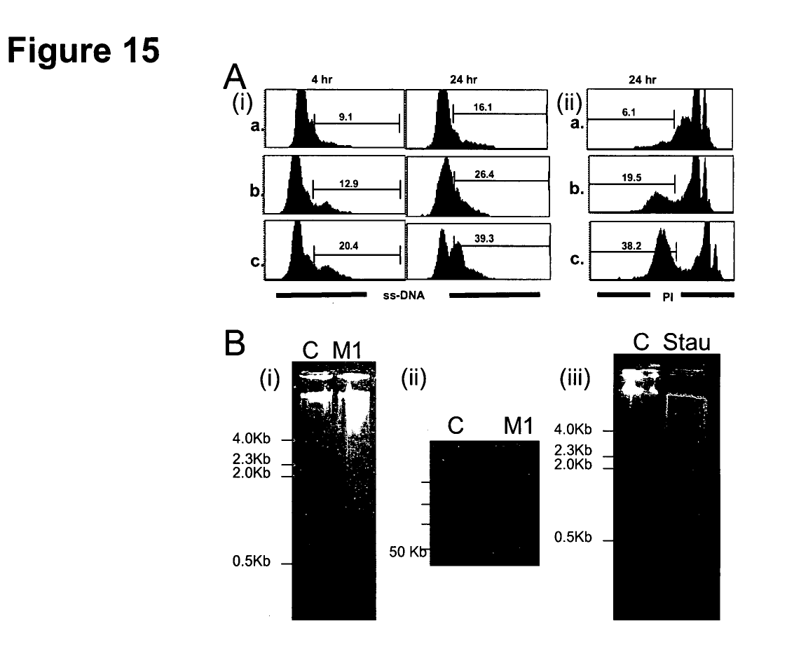

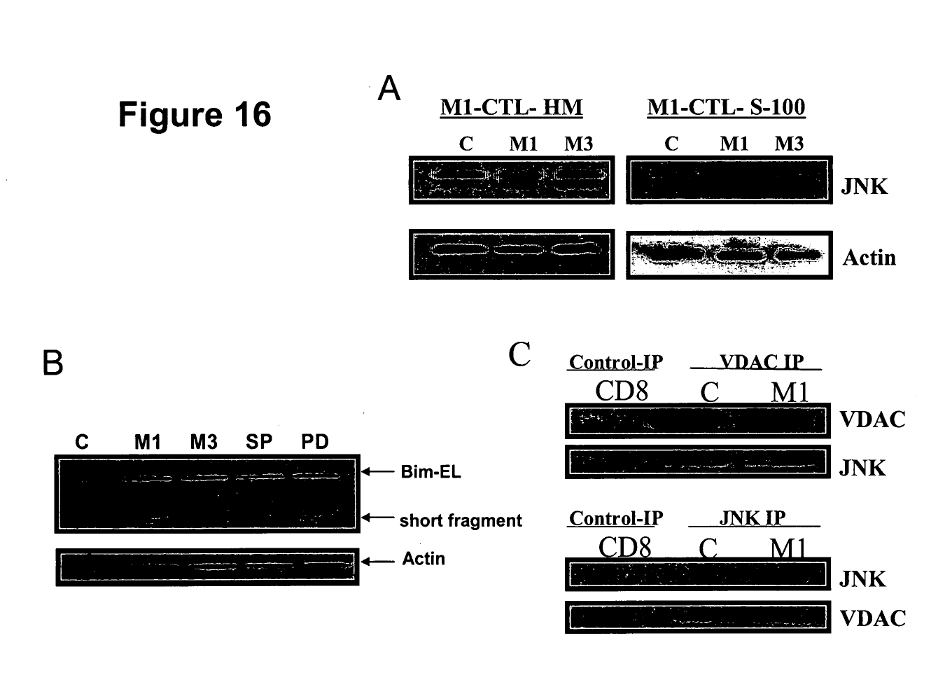

We have found that the AICD in these CTLs is mostly mediated by the activation of a mitochondrial apoptotic machinery characterized by the release of the mitochondria-based apoptosis inducing factor (AIF) without any cytochrome c release. AIF then translocates onto the nuclei and the JNK inhibitor SP600125 blocks the AIF release/translocation (FIG. 14). After translocating onto the nuclei, AIF was found at the ssDNA break sites (FIG. 15). We have also found that JNK and phosphorylated Bim (a BH3-only proapoptotic Bcl-2 family protein) co-localize in the CTL and that a short phosphorylated fragment of Bim, the phosphorylation of which is inhibitable by the JNK inhibitor SP600125, is generated in these CTLs during AICD (FIG. 16). Biochemical analyses showed JNK to be present on mitochondria and to interact with the mitochondrial porin, voltage dependent anion channel (VDAC) (FIG. 16).

While not intending to be limited to any one theory, the applicants believe the data support the following conclusions: First, although controversy exists on the “primacy” between caspases and mitochondria in cellular apoptosis, our data strongly suggests a mitochondrial process in AICD of primary CTLs. Second, our data show, for the first time, the existence of a novel mitochondria-based and caspase-independent process (AIF release and no cytochrome c release) in AICD of primary CTLs. Third, the data implicate JNK in orchestrating the AIF release leading to large scale DNA fragmentation as the mode of death in these cells.

That AIF is a mitochondria-based apoptotic effector protein and upon release from mitochondria, AIF causes large scale DNA breaks leading to apoptosis is now well established. However, the precise mechanism underlying AIF release (and underlying cytochrome c release, for that matter) is not fully understood. A role for JNK in neuronal apoptosis is also well established. JNK as well as Bim have been implicated in T cell apoptosis. Again, the precise mechanism by which JNK participates in cell death is not fully understood. Now that we are able to trace JNK to mitochondrial outer membrane, show the generation of a short phosphorylated Bim fragment apparently by JNK during AICD, and find that JNK physically interacts with VDAC (FIG. 16), JNK may have a role in orchestrating the mitochondrial membrane permeabilization, directly (by interacting with VDAC) or indirectly (through Bcl-2 family intermediaries).

Taken together, our data reveal, for the first time, the existence of a novel mitochondria-based and caspase-independent process (AIF release and no cytochrome c release) in AICD in primary CTLs. The data also implicate JNK in orchestrating the AIF release resulting from mitochondrial membrane permeabilization and/or dysregulation seemingly driven by a direct JNK-VDAC interaction or a JNK-Bcl-2 family pro-apoptotic protein-VDAC interactions. Thus our data provide new insights into the process underlying AICD and offer opportunities to devise ways for interfering with AICD in tumor epitope specific CTLs at several different points so as to orchestrate a more robust and long-lived anti-tumor CTL response induced by active specific vaccination or adoptive therapy. The present invention has utility for treatment of any disease for which the death of CTLs is a factor in the etiology, mechanism or progression of that disease.

In further experiments we have observed that JNK inhibitors such as SP600125 also inhibit/prevent AICD in helper T cells. Thus, JNK inhibitors, when administered alone or in combination with other therapeutics, have utility for the treatment of diseases, including AIDS, for which the death of helper T cells is a factor in the etiology, mechanism or progression of that disease.

### REFERENCES

Each of these documents is hereby incorporated by reference in their entirety.
Adams, J. A., and S. Cory. 1998. The Bcl-2 protein family: arbiters of cell survival. Science 281:1322.
Alderson, M. R., Tough, T. W., Davis-Smith, T., Braddy, S., Falk, B., Schooley, K. A., Goodwin, R. G., Smith, C. A., Ramsdell, F., and Lynch, D. H. Fas ligand mediates activation-induced cell death in human T lymphocytes. J Exp Med, 181: 71-77, 1995.
Ashwell, J. D., R. E. Cunningham, P.D. Noguchi, and D. Hernandez. 1987. Cell growth cycle block of T cell hybridomas upon activation with antigen. J. Exp. Med. 165: 173.
Badovinac V. P., B.B. Poter and J. T. Harty. 2003. Programmed contraction of CD 8+ T cells after infection. Nat. Immunol. 3:619.
Banki, K., E. Hutter, N. J. Gonchoroff, and A. Perd. 1999. Elevation of mitochondrial transmembrane potential and reactive oxygen intermediate levels are early events and occur independently from activation of caspases in Fas signaling. J Immunol. 162:1466.
Brunner, T., Mogil, R. J., LaFace, D., Yoo, N. J., Mahboubi, A., Echeverri, F., Martin, S. J., Force, W. R., Lynch, D. H., Ware, C. F., and et al. Cell-autonomous Fas (CD95)/Fas-ligand interaction mediates activation-induced apoptosis in T-cell hybridomas. Nature, 373: 441-444, 1995.
Budd, R. C. 2001. Activation-induced cell death. Curr. Opin. Immunol. 13:356.
Chakraborty N G, L. Li, J. R. Sporn, S. H. Kurtzman, M. T. Ergin, and B. Mukherji. 1999. Emergence of regulatory CD4+ T cell response to repetitive stimulation with antigen-presenting cells in vitro: implications in designing antigen-presenting cell-based tumor vaccines. J Immunol. 162:5576.
Coulie, P. G., V. Brichard, A. Van Pel, T. Wolfel, J. Schneider, C. Traversari, S. Mattei, E. De Plaen, C. Lurquin, and J. P. Szikora et al. 1994. A new gene coding for a differentiation antigen recognized by autologous cytolytic T lymphocytes on HLA-A2 melanomas. J Exp. Med. 180: 35.
Daugas, E., Susin, S. A., Zamzami, N., Ferri, K. F., lrinopoulou, T., Larochette, N., Prevost, M. C., Leber, B., Andrews, D., Penninger, J., and Kroemer, G. Mitochondrio-nuclear translocation of AIF in apoptosis and necrosis. Faseb J, 14: 729-739, 2000.
Dhein, J., H. Walczak, C. Baumler, K. Debatin, and P. Krammer. 1995. Autocrine T-cell suicide mediated by Apo-1 (Fas/CD95). Nature 373: 438.
Dong, C., R. J. Davis, R. A. Flavell. 2002. MAP kinases in the immune response. Annu Rev Immunol. 20:55.
Frankfurt, O. S., Robb, J. A., Sugarbaker, E. V., and Villa, L. Monoclonal antibody to single-stranded DNA is a specific and sensitive cellular marker of apoptosis. Exp Cell Res, 226: 387-397, 1996.
Grayson, J. M., Laniewski, N. G., Lanier, J. G., and Ahmed, R. Mitochondrial potential and reactive oxygen intermediates in antigen-specific CD8+ T cells during viral infection. J Immunol, 170: 4745-4751, 2003.
Green, D. R. and Reed, J. C. Mitochondria and apoptosis. Science, 281: 1309-1312, 1998.
Green, D. R., Droin, N., and Pinkoski, M. Activation-induced cell death in T cells. Immunol Rev, 193: 70-81, 2003.
Gross, A., Jockel, J., Wei, M. C., and Korsmeyer, S. J. Enforced dimerization of BAX results in its translocation, mitochondrial dysfunction and apoptosis. Embo J, 17: 3878-3885, 1998.
Hildeman, D. A., Mitchell, T., Teague, T. K., Henson, P., Day, B. J., Kappler, J., and Marrack, P. C. Reactive oxygen species regulate activation-induced T cell apoptosis. Immunity, 10: 735-744, 1999.
Hildeman, D. A., Y. Zhu, T. C. Mitchell, J. Kappler, and P. Marrack. 2002. Molecular mechanisms of activated T cell death in vivo. Curr. Opin. Immunol. 14:354.
Houghton, A. N. 1994. Cancer antigens: immune recognition of self and altered self. J. Exp. Med. 180: 1.
Jaattela, M. and Tschopp, J. Caspase-independent cell death in T lymphocytes. Nat Immunol, 4: 416-423, 2003.
Kawakami, Y., S. Eliyahu, K. Sakaguchi, P. F. Robbins, L. Rivoltini, J. R. Yannelli, E. Appella, and S. A. Rosenberg. 1994. Identification of the immunodominant peptides of the MART-1 human melanoma antigen recognized by the majority of HLA-A2-restricted tumor infiltrating lymphocytes. J. Exp. Med. 180: 347.
Kroemer, G. and Martin, S. J. Caspase-independent cell death. Nat Med, 11: 725-730, 2005.
Lenardo, M. J. Interleukin-2 programs mouse alpha beta T lymphocytes for apoptosis. Nature, 353: 858-861, 1991.
Mehrotra, S., Chhabra, A., Chattopadhyay, S., Dorsky, D. I., Chakraborty, N. G., and Mukherji, B. Rescuing melanoma epitope-specific cytolytic T lymphocytes from activation-induced cell death, by SP600125, an inhibitor of JNK: implications in cancer immunotherapy. J Immunol, 173: 6017-6024, 2004.
Mehrotra, S., R. Stevens, R. Zengou, N. G. Chakraborty, L. H. Butterfield, J. S. Economou, D. I. Dorsky, and B. Mukherji. 2003. Regulation of melanoma epitope-specific cytolytic T lymphocyte response by immature and activated dendritic cells, in vitro. Cancer Res 63: 5607.
Mukherji, B., A. Guha, N. G. Chakraborty, M. Sivanandham, A. L. Nashed, J. R. Sporn, and M. T. Ergin. 1989. Clonal analysis of cytotoxic and regulatory T cell responses against human melanoma. J Exp Med. 169:1961.
Mukherji, B., N. G. Chakraborty, S. Yamasaki, T. Okino, H. Yamase, J. R. Sporn, S. K. Kurtzman, M. T. Ergin, J. Ozols, J. Meehan. 1995. Induction of antigen-specific cytolytic T cells in situ in human melanoma by immunization with synthetic peptide-pulsed autologous antigen presenting cells. Proc Natl Acad Sci U S A 92:8078.
Murali-Krishna, K., J. D. Altman, M. Suresh, D. J. Sourdive, A. J. Zajac, J. D. Miller, J. Slansky, and R. Ahmed. 1998. Counting antigen-specific CD8 T cells: a reevaluation of bystander activation during viral infection. Immunity 8:177.
Nagy, G., A. Koncz, and A. Perl. 2003. T cell activation induced mitochondrial hyperpolarization is mediated by Ca2+ and redox dependent production of nitric oxide. J. Immunol. 171:5188.
Pardoll G., Allison J. Cancer immunotherapy: breaking the barriers to harvest the crop. Nat. Med. 10:9, 887-892.
Parmiani, G., C. Castelli, P. Dalerba, R. Mortarini, L. Rivoltini, F. M. Marincola, and A. Anichini. 2002. Cancer immunotherapy with peptide based vaccines: What have we achieved? Where are we going? J. Natl. Cancer Inst. 94: 805.
Perl, A., P. Gergely Jr, G. Nagy, A. Koncz, and K. Banki. 2004. Mitochondrial hyperpolarization: a checkpoint of T-cell life, death and autoimmunity. Trends Immunol. 25:360.
Rincon, M., and G. Pedraza-Alva. 2003. JNK and p38 MAP kinases in CD4+ and CD 8+ T cells. Immunol. Rev. 192:131.
Rosenberg, S. A., Yang, J. C., and Restifo, N. P. Cancer immunotherapy: moving beyond current vaccines. Nat Med, 10: 909-915, 2004.
Schluns, K. S., and L. Lefrancois. 2003. Cytokine control of memory T-cell development and survival. Nat. Rev. Immunol. 3:269.
Shi Y. F., B. Sahai, and D. R. Green. 1989. Cyclosporin A inhibits activation-induced cell death in T-cell hybridomas and thymocytes. Nature. 339: 625.
Shi, Y. F., Bissonnette, R. P., Parfrey, N., Szalay, M., Kubo, R. T., and Green, D. R. In vivo administration of monoclonal antibodies to the CD3 T cell receptor complex induces cell death (apoptosis) in immature thymocytes. J Immunol, 146: 3340-3346, 1991.
Shi, Y. F., Szalay, M. G., Paskar, L., Sahai, B. M., Boyer, M., Singh, B., and Green, D. R. Activation-induced cell death in T cell hybridomas is due to apoptosis. Morphologic aspects and DNA fragmentation. J Immunol, 144: 3326-3333, 1990.
Smith, C. A., Williams, G. T., Kingston, R., Jenkinson, E. J., and Owen, J. J. Antibodies to CD3/T-cell receptor complex induce death by apoptosis in immature T cells in thymic cultures. Nature, 337: 181-184, 1989.
Susin, S. A., Lorenzo, H. K., Zamzami, N., Marzo, I., Snow, B. E., Brothers, G. M., Mangion, J., Jacotot, E., Costantini, P., Loeffler, M., Larochette, N., Goodlett, D. R., Aebersold, R., Siderovski, D. P., Penninger, J. M., and Kroemer, G. Molecular characterization of mitochondrial apoptosis-inducing factor. Nature, 397: 441-446, 1999.
van Parjis, L., and A. K. Abbas. 1998. Homeostasis and self-tolerance: Turning lymphocytes off. Science 280: 243.
Waldmann, T. A., S. Dubois, and Y. Tagaya. 2001. Contrasting roles of IL-2 and IL-15 in the life and death of lymphocytes: implications for immunotherapy. Immunity 14: 105.
Zhang, X., S. Sun, I. Hwang, D. F. Tough, and J. Sprent. 1998. Potent and selective stimulation of memory-phenotype CD 8+ T cells in vivo by IL-15. Immunity 8: 591.
Zheng, L., Fisher, G., Miller, R. E., Peschon, J., Lynch, D. H., and Lenardo, M. J. Induction of apoptosis in mature T cells by tumour necrosis factor. Nature, 377: 348-351, 1995.

1. A method for inhibiting cell death in antigen specific T cells comprising:
providing an antigen specific T cell from a subject;
activating said antigen specific T cell; and
administering an effective amount of a JNK inhibitor, an AIF inhibitor or both to the activated antigen specific T cell.

2. The method of claim 1, wherein the activated antigen specific T cell comprises a CD8⁺ cytotoxic T lymphocyte.

3. The method of claim 1, wherein the antigen specific T cell comprises a cell isolated from a subject and cultured in vitro or ex vivo.

4. The method of claim 1, wherein said activating step is performed by secondary exposure of the T cell with an antigen, incubation in culture with at least one mature DC, or a combination thereof.

5. The method of claim 4, wherein the antigen specific T cell is cultured in media further comprising IL-15 at a concentration of from about 0.1 ng/ml to about 100 ng/ml.

6. The method of claim 1, wherein the JNK inhibitor comprises a pyrazoloanthrone, a pyrazoloanthrone derivative, a nucleic acid or combinations thereof.

7. The method of claim 6, wherein the JNK inhibitor is administered as a pharmaceutically acceptable salt, base, or combination thereof.

8. The method of claim 1, wherein the AIF inhibitor comprises a nucleic acid, pharmaceutically acceptable salts, bases, or combinations thereof.

9. An antigen specific cytotoxic T lymphocyte cell created according to the method of claim 1.

10. A method of treating a disease comprising administering the antigen specific cytotoxic T lymphocyte cell of claim 9 to the subject from which the cell was initially provided.

11. The method of claim 6, wherein the pyrazoloanthrone or derivative is present at a concentration of from about 0.1 μM to about 1 mM.

12. The method of claim 6, wherein the pyrazoloanthrone comprises anthra[1,9-cd]pyrazol-6(2H)-one, derivatives, pharmaceutically acceptable salts, bases, or combinations thereof.

13. A therapeutic composition for improving immunity comprising an effective amount of a pyrazoloanthrone, a pyrazoloanthrone derivative, a nucleic acid complementary to a JNK RNA, a nucleic acid complementary to an AIF RNA, or combinations thereof.

14. The therapeutic composition of claim 13, wherein the nucleic acid specific for JNK or AIF is disposed within a nucleic acid vector adapted for expression in a eukaryotic cell.

15. A method of treating a disease in a subject comprising:
providing at least one antigen specific T cell from the subject;
activating the antigen specific T cell in vitro, wherein an effective amount of a JNK inhibitor, an AIF inhibitor or both is administered to the antigen specific T cell; and
administering the activated T cell to the subject from which it was initially provided.

16. The method of claim 15, wherein the disease is a cancer.

17. The method of claim 15, wherein the disease is an immunological disorder.

18. The method of claim 15 wherein the step of activating the antigen specific T cell comprises performing specific immunization of the antigen specific T cell with a disease specific antigen.

19. The method of claim 15, wherein the JNK inhibitor, AIF inhibitor or both is administered before, during, or after activating the antigen specific T cell.

20. The method of claim 15, wherein the JNK inhibitor comprises a pyrazoloanthrone, pyrazoloanthrone derivative, nucleic acid or combination thereof, and optionally in combination with at least one other active agent.

21. The method of claim 15, wherein the AIF inhibitor comprises a nucleic acid.

22. The method of claim 20 or 21, wherein the nucleic acid comprises an shRNA.

23. The method of claim 20, wherein the pyrazoloanthrone comprises anthra[1,9-cd]pyrazol-6(2H)-one, derivatives, combinations and pharmaceutically acceptable salts thereof.

24. A method for inducing AICD in an antigen specific T cell cell comprising the step of upregulating JNK expression, AIF expression, JNK enzyme activity or combinations thereof.

25. A method for enhancing the immunity of a subject, in vivo, comprising administering an effective amount of an inhibitor of JNK, an inhibitor of AIF or combinations thereof in a pharmaceutically acceptable form to a subject having cancer, an immunological disease or both.

26. The method of claim 25, wherein the JNK inhibitor comprises a pyrazoloanthrone, a pyrazoloanthrone derivative, a nucleic acid or combinations thereof.

27. The method of claim 25, wherein the AIF inhibitor comprises a nucleic acid, pharmaceutically acceptable salts, bases, or combinations thereof.

28. The method of claim 26, wherein the pyrazoloanthrone comprises anthra[1,9-cd]pyrazol-6(2H)-one, derivatives, combinations and pharmaceutically acceptable salts thereof.

29. The method of claim 25 wherein the immunological disease is AIDS.

30. A chemical composition comprising a double stranded (ds) nucleic acid molecule that forms an siRNA and that down regulates expression of a JNK gene via RNA-interference, wherein each strand of the ds nucleic acid molecule is independently about 10 to about 40 nucleotides in length; and wherein one strand of the ds nucleic acid molecule comprises a nucleotide sequence having sufficient complementarity to an RNA of the JNK gene for the ds nucleic acid molecule to cause, directly or indirectly, cleavage of said RNA via RNA-interference.

31. A chemical composition comprising a double stranded (ds) nucleic acid molecule that forms an siRNA and that down regulates expression of an AIF gene via RNA-interference, wherein each strand of the ds nucleic acid molecule is independently about 10 to about 40 nucleotides in length; and wherein one strand of the ds nucleic acid molecule comprises a nucleotide sequence having sufficient complementarity to an RNA of the AIF gene for the ds nucleic acid molecule to cause, directly or indirectly, cleavage of said RNA via RNA-interference.

32. The chemical composition of claims 30, wherein the nucleic acid molecule is an enzymatic nucleic acid.

33. The chemical composition of claim 31, wherein the siRNA is generated from an shRNA precursor.

34. The chemical composition of claim 32, wherein the enzymatic nucleic acid comprises a modified nucleotide.

35. The chemical composition of claims 30, further comprising a pharmaceutically acceptable carrier or diluent.

36. The chemical compositions of claim 30, wherein the double stranded nucleic acid molecule is contained in an nucleic acid vector operably linked with one or more DNA regulatory elements.

37. The chemical composition of claim 36, wherein the nucleic acid vector comprising the double stranded nucleic acid molecule is contained within a cell.

38. The chemical composition of claims 30, further comprising an effective amount of a pyrazoloanthrone or pyrazoloanthrone derivative.

39. The chemical composition of claim 30, wherein the ds nucleic acid comprises a strand of nucleotides having at least 85% homology to SEQ ID NO: 5 or SEQ ID NO:6.

40. The chemical composition of claim 31, wherein the ds nucleic acid comprises a strand of nucleotides having at least 85% homology to SEQ ID NO:7.
